(19) United States
(12) Patent Application Publication
(10) Pub. No.: US 2025/0354155 A1
(43) Pub. Date: Nov. 20, 2025

(54) METHODS OF TREATING X-LINKED GENETIC DISEASES

(71) Applicant: YISSUM RESEARCH DEVELOPMENT COMPANY OF THE HEBREW UNIVERSITY OF JERUSALEM LTD., Jerusalem (IL)

(72) Inventors: Rotem KARNI, Mevaseret Zion (IL); Ela ELYADA, Mevaseret Zion (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/872,179

(22) PCT Filed: Jun. 7, 2023
(86) PCT No.: PCT/IL2023/050589

§ 371 Date: Dec. 5, 2024

Related U.S. Application Data
(60) Provisional application No. 63/349,647, filed on Jun. 7, 2022.

(51) Int. Cl. C12N 15/113

(52) U.S. Cl. CPC C12N 15/1138; C12N 2310/11; C12N 2310/315; C12N 2310/321; C12N 2310/341

(57) ABSTRACT

Methods of treating a neurodevelopmental disorder caused by a mutation in HNRNPH2 or PCDH19, comprising downregulating expression of HNRNPH2 or PCDH19 in cells of the brain of a subject are provided. Antisense oligonucleotides useful in performing the methods of the invention are also provided.

### CROSS REFERENCE TO RELATED APPLICATIONS

This application is a National Phase of PCT Patent Application No. PCT/IL2023/050589 having International filing date of Jun. 7, 2023, which claims the benefit of priority under 35 U.S.C. § 119 (e) of U.S. Provisional Patent Application No. 63/349,647 filed on Jun. 7, 2022, the contents of which are all incorporated by reference as if fully set forth herein in their entirety.

### REFERENCE TO AN ELECTRONIC SEQUENCE LISTING

The contents of the electronic sequence listing (HUJI-P-088-US.xml; Size: 223,658 bytes; and Date of Creation: Dec. 29, 2024) is herein incorporated by reference in its entirety.

### FIELD OF INVENTION

The present invention is in the field of X-linked genetic diseases.

### BACKGROUND OF THE INVENTION

Point mutations in the genes HNRNPH2 and PCDH19 each cause a rare childhood neurodevelopmental disease. Mutations in HNRNPH2 cause Mental retardation, X-linked, syndromic, Bain-type (MRXSB). Mutations in PCDH19 cause Early Infantile Epileptic Encephalopathy type 9 (EIEE-9), also known as Epilepsy in Females with Mental Retardation (EFMR). These diseases are characterized by developmental and intellectual retardation, muscle hypotonia, autism spectrum disorders and severe epileptic seizures. Both genes are located on the X chromosome, and the disease is manifested in a heterozygous setting (in females) or mosaic expression (in males). The arbitrary process of X inactivation in females, likewise the mosaic expression of the mutation in males, creates a mixture of cells; some with intact protein and others with mutated protein, a situation which is believed to be the driver of the neurological phenotype. Methods of treating these rare, devastating, developmental disorders greatly needed.

### SUMMARY OF THE INVENTION

The present invention provides methods of treating a neurodevelopmental disorder caused by a mutation in HNRNPH2 or PCDH19, comprising downregulating expression of HNRNPH2 or PCDH19 in cells of the brain. Antisense oligonucleotides useful in performing the methods of the invention are also provided.

According to a first aspect, there is provided a method of treating a neurodevelopmental disorder caused by a mutation in heterogenous nuclear ribonucleoprotein H2 (HNRNPH2) in a subject in need thereof, the method comprising administering to the brain of the subject an antisense oligonucleotide that binds to wild-type and mutant HNRNPH2 and downregulates expression of both but does not bind to HNRNPH1 and downregulate expression, thereby treating a neurodevelopmental disorder caused by a mutation in HNRNPH2.

According to some embodiments, the neurodevelopmental disorder is Mental retardation, X-linked, syndromic, Bain-type (MRXSB).

According to some embodiments, the antisense oligonucleotide is 100% complementary to an HNRNPH2 mRNA but comprises at least two mismatches to an HNRNPH1 mRNA.

According to some embodiments, the HNRNPH2 mRNA comprises or consists of SEQ ID NO: 1 and the HNRNPH1 mRNA comprises or consists of SEQ ID NO: 83.

According to some embodiments, the antisense oligonucleotide is reverse complementary to a sequence from nucleotide 1480-2040 of SEQ ID NO: 1.

According to some embodiments, the antisense oligonucleotide comprises or consists of a sequence selected from SEQ ID NO: 9, 15, 18, and 20-21.

According to another aspect, there is provided a method of treating a neurodevelopmental disorder caused by a mutation in protocadherin 19 (PCDH19) in a subject in need thereof, the method comprising administering to the brain of the subject an antisense oligonucleotide comprising or consisting of a sequence selected from SEQ ID NO: 23-24, 26-27, 29, 31-33, and 35-41.

According to some embodiments, the antisense oligonucleotide comprises or consists of SEQ ID NO: 35 or 36

According to some embodiments, the neurodevelopmental disorder is selected from Early Infantile Epileptic Encephalopathy type 9 (EIEE-9) and Epilepsy in Females with Mental Retardation (EFMR).

According to some embodiments, the neurodevelopmental disorder is characterized by epilepsy, autism spectrum disorder (ASD), or both.

According to some embodiments, the mutation does not produce a dominant loss of function that renders a wild-type allele of the single gene non-functional or with a reduced function.

According to some embodiments, the subject comprises brain cells that express a wild-type allele of the single gene and brain cells that express a mutant allele of the single gene.

According to some embodiments, the downregulating comprises a reduction of at least 80% in expression.

According to some embodiments, expression is selected from mRNA expression, protein expression and both.

According to some embodiments, the antisense oligonucleotide comprises between 12 and 30 bases.

According to some embodiments, the antisense oligonucleotide comprises a chemically modified backbone, at least one non-natural nucleotide, both DNA and RNA bases or a combination thereof.

According to some embodiments, the antisense oligonucleotide comprises a chemically modified backbone comprising phosphorothioate (PS) linkages.

According to some embodiments, the antisense oligonucleotide comprises a DNA core flanked both 5′ and 3′ by RNA bases.

According to some embodiments, the RNA bases comprise a 2′-MOE modification.

According to some embodiments, the method comprises 10 DNA bases flanked by 5 RNA bases 5′ and 5 RNA bases 3′.

According to some embodiments, the antisense oligonucleotide is a GAPmer.

According to some embodiments, the antisense oligonucleotide comprises at least 5 consecutive DNA bases reverse complementary to an mRNA of HNRNPH2 or PCDH19, such that hybridization of the DNA bases to the mRNA induces RNase H mediated cleavage and degradation of the mRNA.

According to some embodiments, the antisense oligonucleotide is specific to HNRNPH2 or PCDH19 and does not substantially bind to an mRNA of any other gene.

According to another aspect, there is provided an antisense oligonucleotide capable of binding to an mRNA of HNRNPH2 and inducing degradation of the mRNA and comprising a nucleotide sequence selected from SEQ ID NO: 3-5, 7-18, and 20-21.

According to some embodiments, the antisense oligonucleotide is specific to HNRNPH2 and does not significantly bind to HNRNPH1 and comprises or consists of a sequence selected from SEQ ID NO: 3-5, 7-10, 12-13, 15, 18, and 20-21.

According to some embodiments, the antisense oligonucleotide comprises or consists of a sequence selected from SEQ ID NO: 9, 15, 18, and 20-21.

According to another aspect, there is provided an antisense oligonucleotide capable of binding to an mRNA of PCDH19 and inducing degradation of the mRNA and comprising a nucleotide sequence selected from SEQ ID NO: 23-24, 26-27, 29, 31-33 and 35-41.

According to some embodiments, the antisense oligonucleotide comprises a chemically modified backbone, at least one non-natural nucleotide, both DNA and RNA bases or a combination thereof.

According to some embodiments, the antisense oligonucleotide comprises a chemically modified backbone comprising phosphorothioate (PS) linkages.

According to some embodiments, the antisense oligonucleotide comprises a DNA core flanked both 5′ and 3′ by RNA bases.

According to some embodiments, the RNA bases comprise a 2′-MOE modification.

According to some embodiments, the antisense oligonucleotide comprises 10 DNA bases flanked by 5 RNA bases 5′ and 5 RNA bases 3′.

According to some embodiments, the antisense oligonucleotide is a GAPmer.

According to some embodiments, the antisense oligonucleotide is specific to HNRNPH2 or PCDH19 and does not substantially bind to an mRNA of any other gene.

According to another aspect, there is provided a pharmaceutical composition comprising an antisense oligonucleotide of the invention and a pharmaceutically acceptable carrier excipient or adjuvant.

According to some embodiments, the pharmaceutical composition is for use in a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C. Screen for HNRNPH2 GAPmers in human cells. (1A-1B) Quantitative Real-Time PCR (qRT-PCR) of HNRNPH2 and HNRNPH1 in HEK293T cells (1A) and OVCA433 cells (1B) that were transfected with 50 nM of each GAPmer, for 48 hours. siGFP and GAPmers 21, 231, 1271 and 2261 serve as negative controls. siH2 serves as a positive control. (1C) Western blot (WB) analysis of HNRNPH2 in Lan2 neuroblastoma cells expressing Flag-HNRNPH2 cDNA, that were transfected with different HNRNPH2 GAPmers at 50 nM for 48 hours. u.t., untreated; siH2 serves as a positive control; control GAPmer 1271 serves as a negative control. β-catenin serves as a gel loading control.

FIGS. 2A-2C. Screen for PCDH19 GAPmers in human cells. (2A-2B) qRT-PCR of PCDH19 in HEK293T cells (2A) and OVCA433 cells (2B) that were transfected with 50 nM of each GAPmer for 48 hours. siGFP and GAPmers 201, 2671, 5221 and 8401 serve as negative controls. siPCDH serves as a positive control. (2C) WB analysis of PCDH19 in OVCA433 cells transfected with PCDH19 GAPmers. u.t., untreated; siGFP and cont GAPmers serve as negative controls; siPCDH serve as a positive control. Tubulin serves as a gel loading control.

FIGS. 3A-3C. GAPmers efficiency in mouse embryonic fibroblasts. (3A) qRT-PCR of Hnrnph2 and Hnrnph1 in mouse embryonic fibroblasts (MEFs) transfected with 50 nM of each GAPmer for 24 hours. 231 is a control GAPmer. (3B) qRT-PCR of Pcdh19 in MEFs transfected with 50 nM of each GAPmer for 48 hours. 201 is a control GAPmer. (3C) WB analysis of PCDH19 in MEFs transfected with PCDH19 GAPmers. u.t., untreated; Cont 201 GAPmer serves as a negative control. Tubulin serves as a gel loading control.

FIGS. 4A-4D. GAPmer-mediated depletion of HNRNPH2 and HNRNPH1 alters splicing. (4A-4B) Reverse Transcriptase PCR (RT-PCR) analysis using LabChip (for HEK293T) or standard gel electrophoresis (for MDA-MB-231) for exon 4 of MAST1 (4A) and exon 11a of STK36 (4B). (4C) RT-PCR analysis using gel electrophoresis for exon 16 of MADD in HEK293T and MDA-MB-231 cells. (4D) RT-PCR analysis using gel electrophoresis for STK36 exon 11a, MADD exon 16, LSM14B exon 8 and SAT1 exon 4 in MDA-MB-231 cells constitutively expressing empty vector, ectopic WT HNRNPH2 or ectopic mutant (MUT) HNRNPH2 and treated with different GAPmers for 48 hours. Actin is a loading control.

FIGS. 5A-5E. GAPmer-mediated inhibition in vivo. (5A) qRT-PCR of Hnrnph2 and Hnrnph1 in brain tissue from NOD-SCID mice injected ICV with 100 μg of HNRNPH2 GAPmer 1500 or PBS and sacrificed at day 14 after injection (n=3). (5B) qRT-PCR of Hnrnph2 and Hnrnph1 in brain tissue from NOD-SCID mice injected ICV with 50 μg or 25 μg of HNRNPH2 GAPmer 1500, or PBS, and sacrificed at day 7 after injection (n=3). Expression is normalized to Hprt. Shown is Mean±SEM. (5C) qRT-PCR of Pcdh19 in brain tissue from C57BL/6 mice injected ICV through cannulas 3 times a week for two weeks with 50 μg per injection of PCDH19 GAPmer 4190, sense GAPmer 8401 or PBS (to a total of 300 μg) and sacrificed 14 days after the first injection (n=10/group). Expression is normalized to Hprt and Ndufb9. Shown is Mean±SEM, Unpaired t-test: *0.0232, **0.0113, ***0.0002. (5D) Bar graph of the average of all the mice that received each treatment. (5E) WB analysis of PCDH19 in mouse brains from 5C. Tubulin is a loading control.

### DETAILED DESCRIPTION OF THE INVENTION

The present invention, in some embodiments, provides methods of treating X-linked genetic neurodevelopmental disorders caused by a mutation in a single gene on the X chromosome, the method comprising downregulating expression of the single gene on the X chromosome in cells of the brain. Antisense oligonucleotides useful in performing the methods of the invention are also provided.

By a first aspect, there is provided an oligonucleotide capable of binding to an mRNA of a target gene.

In some embodiments, the target gene is associated with an X-linked genetic disorder. In some embodiments, mutation of the target gene is associated with an X-linked genetic disorder. In some embodiments, mutation of the target gene causes an X-linked genetic disorder. In some embodiments, the target gene is heterogenous nuclear ribonucleoprotein H2 (HNRNPH2). In some embodiments, the target gene is protocadherin 19 (PCDH19).

HNRNPH2 belongs to the family of ubiquitously expressed heterogeneous nuclear ribonucleoproteins (hnRNPs). This family of proteins is known to bind RNA and in particular pre-mRNAs and influence processing, splicing and transport among other functions. HNRNPH2 is located on the X chromosome in humans and there are several splice variants of HNRNPH2. In some embodiments, the HNRNPH2 is mammalian HNRNPH2. In some embodiments, the mammal is a human. Human HNRNPH2 is disclosed in Entrez gene number 3188. The human protein sequence is provided in UniProt entry P55795. The human HNRNPH2 mRNA is provided in RefSeq entries NM_019597 and NM_001032393. In some embodiments, the HNRNPH2 mRNA comprises or consists of

(SEQ ID NO: 1)
actcgcttacgtgagcagaagtagttctggtcgtcgtctaccgtctcgctatagccgtttgagggaagaaggaggaaaattacccgg
tatcgttagagctacaccaaaattgcattgagccaaacttgccaccaagagcccaacaatcaccatgatgctgagcacggaaggc
agggaggggttcgtggtgaaggtcaggggcctaccctggtcctgctcagccgatgaagtgatgcgcttcttctctgattgcaagat
ccaaaatggcacatcaggtattcgtttcatctacaccagagaaggcagaccaagtggtgaagcatttgttgaacttgaatctgaaga
ggaagtgaaattggctttgaagaaggacagagaaaccatgggacacagatacgttgaagtattcaagtctaacagtgttgaaatgg
attgggtgttgaagcatacaggtccgaatagccctgatactgccaacgatggcttcgtccggcttagaggactcccatttggctgta
gcaaggaagagattgttcagttcttttcagggttggaaattgtgccaaatgggatgacactgccagtggactttcaggggcgaagca
caggggaagcctttgtgcagtttgcttcacaggagatagctgagaaggccttaaagaaacacaaggaaagaatagggcacaggt
acattgagatcttcaagagtagccgagctgaagttcgaacccactatgatccccctcgaaagctcatggctatgcagcggccaggt
ccctatgataggccgggggctggcagagggtataatagcattggcagaggagctgggtttgaaaggatgaggcgtggtgcctat
ggtggagggtatggaggctatgatgactatggtggctataatgatggatatggctttgggtctgatagatttggaagagacctcaatt
actgtttttcaggaatgtctgatcatagatacggagatggtgggtccagtttccagagcaccacagggcactgtgtacacatgaggg
ggttaccttacagagccactgagaatgatatttataatttcttctcacctcttaatcccatgagagtacatattgaaattggacccgatgg
cagagttaccggtgaggcagatgttgaatttgctactcatgaagatgctgtggcagctatggcaaaagacaaagctaatatgcaaca
cagatatgtggagctcttcttaaattctactgcaggaacaagtgggggtgcttacgatcacagctatgtagaactttttttgaattctaca
gcaggggcaagtggtggcgcttatggtagccaaatgatgggagggatgggcttatccaaccagtctagttatggaggtcctgcta
gccagcagctgagtggtggttatggaggtggttatggtggtcagagcagtatgagtggatatgaccaagttctgcaggaaaactcc
agtgactatcagtcaaaccttgcttaggtagagaaggagcactaaatagctactccagatataaaagctgtacatttgtgggagttga
atagaatgggagggatgtttagtatatccagtatgattggtaaatgggaaatataattgattctgatcactcttggtcagcttctctttcttt
atctttctttctccttttttaagaaaacgagttaagtttaacagttttgcattacaggcttgtgattcatgcttactgtaaagtggaagttgag
attattttaaaacttcaagctcagtaattttgaacactgaaacattcatctaggacataataacaaagttcagtattgaccataactgttaa
aacaatttttagctttcctcaagttagttatgttgtaggagtgtacctaagcagtaagcgtatttaggttaatgcagtttcacttatgttaaat
gttgctcttataccacaaatacattgaaaacttcggatgcatgttgagaaacatgcctttctgtaaaactcaaatataggagctgtgtctac
gattcaaagtgaaaacatttggcatgtttgttaattctagctttttggtttaatatcctgtaaggcacgtgagtgtacacttttttttttttta
aggatacgggacaattttaagatgtaataccaatactttagaagtttggtcgtgtcgtttgtatgaaaatctgaggctttggtttaaatctt
tccttgtattgtgatttccatttagatgtattgtactaagtgaaacttgttaaataaatcttccttttaaaaactgga.
The human HNRNPH2 protein is provided in RefSeq entries NP_062543 and NP_001027565.

PCDH19 belongs to the family of protocadherins (Pcdhs). This family of proteins is involved in cell-adhesion and often engage in homophilic interactions with other protocaderins. PCDH19 is located on the X chromosome in humans and there are several splice variants of PCDH19. In some embodiments, the PCDH19 is mammalian PCDH19. In some embodiments, the mammal is a human. Human PCDH19 is disclosed in Entrez gene number 57526. The human protein sequence is provided in UniProt entry Q8TAB3. The human PCDH19 mRNA is provided in RefSeq entries NM_001184880, NM_020766, NM 001105243 and XM 011530997. In some embodiments, the PCDH19 mRNA comprises or consists of SEQ ID NO: 2. The human PCDH19 protein is provided in RefSeq entries NP_001171809, NP_065817, NP_001098713 and XP_011529299.

In some embodiments, the oligonucleotide is an antisense oligonucleotide. In some embodiments, the oligonucleotide is capable of binding the target mRNA. In some embodiments, the oligonucleotide is reverse complementary to the mRNA. In some embodiments, the mRNA is the target mRNA. In some embodiments, the target is HNRNPH2. In some embodiments, the target is PCDH19. In some embodiments, binding is specifically binding. In some embodiments, specific comprises not significantly binding another mRNA. In some embodiments, specific binding comprises at least a 2, 3, 5, 10, 15, 20, 25, 30, 40, 50, 60, 70, 80, 90 or 100 times greater binding to the target mRNA than to any other mRNA. Each possibility represents a separate embodiment of the invention. In some embodiments, the other mRNA is an HNRNPH1 mRNA. In some embodiments, the other mRNA is another protocadherin. In some embodiments, the other mRNA is a cadherin. In some embodiments, the oligonucleotide is reverse complementary to the target mRNA. In some embodiments, reverse complementary is perfectly complementary. In some embodiments, reverse complementary is at least 80, 85, 90, 93, 95, 97, 99 or 100% complementary. Each possibility represents a separate embodiment of the invention. In some embodiments, the oligonucleotide does not comprise any mismatch to the target mRNA. In some embodiments, the oligonucleotide comprises at most 1, 2, 3, 4, or 5 mismatches to the target mRNA. Each possibility represents a separate embodiment of the invention.

In some embodiments, the oligonucleotide does not bind to HNRNPH1. In some embodiments, the HNRNPH1 is mammalian HNRNPH1. In some embodiments, the mammal is a human. Human HNRNPH1 is disclosed in Entrez gene number 3187. The human protein sequence is provided in UniProt entry P31943. The human HNRNPH1 mRNA is provided in RefSeq entries NM_005520, NM_001257293 and NM 001363572. In some embodiments, the HNRNPH2 mRNA comprises or consists of

(SEQ ID NO: 83)
catttcgtcttagccacgcagaagtcgcgtgtctagtttgtttcgacgccggaccgcgtaagagacgatgatgttgggcacggaag
gtggagagggattcgtggtgaaggtccggggcttgccctggtcttgctcggccgatgaagtgcagaggtttttttctgactgcaaaa
ttcaaaatggggctcaaggtattcgtttcatctacaccagagaaggcagaccaagtggcgaggcttttgttgaacttgaatcagaag
atgaagtcaaattggccctgaaaaaagacagagaaactatgggacacagatatgttgaagtattcaagtcaaacaacgttgaaatg
gattgggtgttgaagcatactggtccaaatagtcctgacacggccaatgatggctttgtacggcttagaggacttccctttggatgta
gcaaggaagaaattgttcagttcttctcagggttggaaatcgtgccaaatgggataacattgccggtggacttccaggggaggagt
acgggggaggccttcgtgcagtttgcttcacaggaaatagctgaaaaggctctaaagaaacacaaggaaagaatagggcacagg
tatattgaaatctttaagagcagtagagctgaagttagaactcattatgatccaccacgaaagcttatggccatgcagcggccaggtc
cttatgacagacctggggctggtagagggtataacagcattggcagaggagctggctttgagaggatgaggcgtggtgcttatggt
ggaggctatggaggctatgatgattacaatggctataatgatggctatggatttgggtcagatagatttggaagagacctcaattact
gtttttcaggaatgtctgatcacagatacggggatggtggctctactttccagagcacaacaggacactgtgtacacatgcggggat
taccttacagagctactgagaatgacatttataattttttttcaccgctcaaccctgtgagagtacacattgaaattggtcctgatggcag
agtaactggtgaagcagatgtcgagttcgcaactcatgaagatgtgtggcagctatgtcaaaagacaaagcaaatatgcaacaca
gatatgtagaactcttcttgaattctacagcaggagcaagcggtggtgcttacgaacacagatatgtagaactcttcttgaattctaca
gcaggagcaagcggtggtgcttatggtagccaaatgatgggaggcatgggcttgtcaaaccagtccagctacgggggcccagc
cagccagcagctgagtgggggttacggaggcggctacggtggccagagcagcatgagtggatacgaccaagttttacaggaaa
actccagtgattttcaatcaaacattgcataggtaaccaaggagcagtgaacagcagctactacagtagtggaagccgtgcatctat
gggcgtgaacggaatgggagggttgtctagcatgtccagtatgagtggtggatggggaatgtaattgatcgatcctgatcactgact
cttggtcaaccttttttttttttttttttttttctttaagaaaacttcagtttaacagtttctgcaatacaagcttgtgatttatgcttactctaagtg
gaaatcaggattgttatgaagacttaaggcccagtatttttgaatacaatactcatctaggatgtaacagtgaagctgagtaaactata
actgttaaacttaagttccagcttttctcaagttagttataggatgtacttaagcagtaagcgtatttaggtaaaagcagttgaattatgtt
aaatgttgccctttgccacgttaaattgaacactgttttggatgcatgttgaaagacatgcttttatttttttgtaaaacaatataggagctg
tgtctactattaaaagtgaaacattttggcatgtttgttaattctagtttcatttaataacctgtaaggcacgtaagtttaagcttttttttttttt
aagttaatgggaaaaatttgagacgcaataccaatacttaggattttggtcttggtgtttgtatgaaattctgaggccttgatttaaatcttt
cattgtattgtgatttccttttaggtatattgcgctaagtgaaacttgtcaaataaatcctccttttaaaaactgca.
The human HNRNPHI protein is provided in RefSeq entries NP_005511,
NP_001244222, NP_001350501, NP_001351154 and NP_001351155.

In some embodiments, the oligonucleotide induces degradation of the mRNA to which it binds. In some embodiments, the oligonucleotide reduces half-life in the cytoplasm of the mRNA to which it binds. In some embodiments, the oligonucleotide reduces protein expression from the mRNA to which it binds. In some embodiments, the oligonucleotide reduces translation of the mRNA to which it binds. In some embodiments, the oligonucleotide inhibits translation of the mRNA to which it binds. In some embodiments, the mRNA to which it binds is the target mRNA. Inhibitory antisense oligonucleotides are well known in the art and the oligonucleotide of the invention is such a molecule as it can reduce protein expression from the mRNA in any way known in the art. In some embodiments, a specific oligonucleotide does not significantly inhibit translation of another mRNA. In some embodiments, a specific oligonucleotide does not significantly degrade another mRNA. In some embodiments, the oligonucleotide reduces expression of mRNA to which it binds. In some embodiments, reduces is downregulates.

In some embodiments, the oligonucleotide binds to a wild-type mRNA. In some embodiments, the oligonucleotide binds to a mutant mRNA. In some embodiments, binds to is hybridizes to. In some embodiments, binds to is reverse complementary to. In some embodiments, the oligonucleotide does not bind at the mutation. In some embodiments, the oligonucleotide is not reverse complementary to a region comprising the mutation. In some embodiments, the oligonucleotide binds a region that does not comprise the mutation. In some embodiments, the oligo nucleotide is not specific to a mutant allele or a mutant mRNA. In some embodiments, the oligonucleotide binds an mRNA of the target gene regardless of the presence or absence of a mutation.

In some embodiments, the oligonucleotide downregulates expression of a wild-type mRNA. In some embodiments, the oligonucleotide downregulates expression of a mutant mRNA. In some embodiments, downregulates expression of is inhibits expression. In some embodiments, the oligo nucleotide is not specific to downregulate a mutant allele or a mutant mRNA. In some embodiments, the oligonucleotide downregulates expression of an mRNA of the target gene regardless of the presence or absence of a mutation.

In some embodiments, the oligonucleotide binds HNRNPH2 and not HNRNPH1. In some embodiments, the oligonucleotide hybridizes HNRNPH2 and not HNRNPH1. In some embodiments, the oligonucleotide is reverse complementary to HNRNPH2 and not HNRNPH1. In some embodiments, the oligonucleotide does not bind HNRNPH1. In some embodiments, the oligonucleotide is not reverse complementary to a region within HNRNP1. In some embodiments, the oligonucleotide binds a region present in HNRPH2 and not present in HNRNPH1. In some embodiments, the oligonucleotide downregulates expression of HNRNPH2 and not HNRNPH1. In some embodiments, the oligonucleotide inhibits expression of HNRNPH2 and not HNRNPH1. In some embodiments, the oligonucleotide does not down regulate expression of HNRNPH1. In some embodiments, the oligonucleotide inhibits expression of HNRNP1. In some embodiments, the oligonucleotide is 100 complementary to HNRNPH2 and comprises at least 1 mismatch to HNRNPH1. In some embodiments, at least 1 is a plurality of mismatches. In some embodiments, at least 1 is at least 2.

In some embodiments, the oligonucleotide is reverse complementary to a sequence from a region in HNRNPH2 with low complementarity to HNRNPH1. In some embodiments, the oligonucleotide is reverse complementary to a sequence with a target region of HNRNPH2. In some embodiments, the region is from nucleotide 1411-2040 of SEQ ID NO: 1. In some embodiments, the region is from nucleotide 1480-2040 of SEQ ID NO: 1. In some embodiments, the region is from nucleotide 1500-2040 of SEQ ID NO: 1. In some embodiments, the region is from nucleotide 1681-2040 of SEQ ID NO: 1. In some embodiments, the region is from nucleotide 1850-2040 of SEQ ID NO: 1. In some embodiments, the region is from nucleotide 1870-2040 of SEQ ID NO: 1. In some embodiments, the region is from nucleotide 1411-2260 of SEQ ID NO: 1. In some embodiments, the region is from nucleotide 1480-2260 of SEQ ID NO: 1. In some embodiments, the region is from nucleotide 1500-2260 of SEQ ID NO: 1. In some embodiments, the region is from nucleotide 1681-2260 of SEQ ID NO: 1. In some embodiments, the region is from nucleotide 1850-2260 of SEQ ID NO: 1. In some embodiments, the region is from nucleotide 1870-2260 of SEQ ID NO: 1.

In some embodiments, the oligonucleotide modulates splicing of a gene that is a target of the mRNA to which is binds. In some embodiments, splicing of a gene is splicing of a mRNA transcribed from the gene. In some embodiments, a target of the mRNA is a target of the protein produced by the mRNA. In some embodiments, the oligonucleotide modulates splicing of a gene that is a target of HNRNPH2. In some embodiments, the oligonucleotide modulates splicing of a gene that is a target of mutant HNRNPH2. In some embodiments, the oligonucleotide does not modulate splicing of a gene that is a target of HNRNPH1. In some embodiments, the oligonucleotide modulates splicing of a gene that is a target of HNRNPH2 and HNRNPH1. In some embodiments, the oligonucleotide modulates splicing of a gene that is a target of HNRNPH2 and not HNRNPH1. In some embodiments, the oligonucleotide modulates HNRNPH2 mediated splicing. In some embodiments, the oligonucleotide does not modulate HNRNPH1 mediated splicing. Without being bound to any particular explanation, it will be understood by a skilled artisan that downregulation of HNRNPH2 will result in a reduced effect of the protein on splicing. Thus, if HNRNPH2 is downregulated its effect on splicing will be reduced, whereas HNRNPH1 which is not downregulated will function normally on splicing and indeed may compensate for the loss of HNRNPH2. Thus, even though wild-type and mutant HNRNPH2 will be reduced, HNRNPH1 will compensate for the lost HNRNPH2 and since the HNRNPH1 is not mutated (as HNRNPH2 is) it will restore normal/healthy splicing. In some embodiments, the oligonucleotide modulates splicing of a gene that is a target of PCDH19. In some embodiments, the oligonucleotide modulates splicing of a gene that is a target of mutant PCDH19. In some embodiments, modulates is increases. In some embodiments, modulates is decreases. In some embodiments, modulates is alters. In some embodiments, modulates is increases skipping. In some embodiments, modulates is decreases skipping. In some embodiments, modulates is increases inclusion. In some embodiments, modulates is decreases inclusion.

In some embodiments, a target of HNRNPH2 is selected from MAP Kinase Activating Death Domain (MADD), Protein LSM14 homolog B (LSM14B), Serine/Threonine Kinase 36 (STK36), Spermidine/spermine N1-acetyltransferase 1 (SAT1) and Microtubule Associated Serine/Threonine Kinase 1 (MAST1). In some embodiments, a target of HNRNPH2 and HNRNPH1 is selected from MADD, STK36 and MAST1. In some embodiments, a target of HNRNPH2 and not HNRNPH1 is selected from SATI and LSM14B. In some embodiments, the antisense oligonucleotide affects the splicing of MADD exon 16. In some embodiments, splicing of MADD is splicing of exon 16 of MADD. In some embodiments, modulates is increases skipping of exon 16 of MADD. In some embodiments, modulates is decreases skipping of exon 16 of MADD. In some embodiments, modulates is increases inclusion of exon 16 of MADD. In some embodiments, modulates is decreases inclusion of exon 16 of MADD. In some embodiments, the antisense oligonucleotide affects the splicing of LSM14B exon 8. In some embodiments, splicing of LSM14B is splicing of exon 8 of LSM14B. In some embodiments, modulates is increases skipping of exon 8 of LSM14B. In some embodiments, modulates is decreases skipping of exon 8 of LSM14B. In some embodiments, modulates is increases inclusion of exon 8 of LSM14B. In some embodiments, modulates is decreases inclusion of exon 8 of LSM14B. In some embodiments, the antisense oligonucleotide affects the splicing of STK36 exon 11A. In some embodiments, splicing of STK36 is splicing of exon 11A of STK36. In some embodiments, modulates is increases skipping of exon 11A of STK36. In some embodiments, modulates is decreases skipping of exon 11A of STK36. In some embodiments, modulates is increases inclusion of exon 11A of STK36. In some embodiments, modulates is decreases inclusion of exon 11A of STK36. In some embodiments, the antisense oligonucleotide affects the splicing of SATI exon 4. In some embodiments, splicing of SATI is splicing of exon 4 of SAT1. In some embodiments, modulates is increases skipping of exon 4 of SAT1. In some embodiments, modulates is decreases skipping of exon 4 of SAT1. In some embodiments, modulates is increases inclusion of exon 4 of SAT1. In some embodiments, modulates is decreases inclusion of exon 4 of SAT1. In some embodiments, the antisense oligonucleotide affects the splicing of MAST1 exon 4. In some embodiments, splicing of MAST1 is splicing of exon 4 of MAST1. In some embodiments, modulates is increases skipping of exon 4 of MAST1. In some embodiments, modulates is decreases skipping of exon 4 of MAST1. In some embodiments, modulates is increases inclusion of exon 4 of MAST1. In some embodiments, modulates is decreases inclusion of exon 4 of MAST1.

In some embodiments, the oligonucleotide comprises a nucleotide sequence disclosed in Table 1. In some embodiments, the oligonucleotide comprises a nucleotide sequence selected from SEQ ID NO: 3-22. In some embodiments, the oligonucleotide consists of a nucleotide sequence selected from SEQ ID NO: 3-22. In some embodiments, the oligonucleotide comprises a nucleotide sequence selected from SEQ ID NO: 3-5, 7-18, and 20-21. In some embodiments, the oligonucleotide consists of a nucleotide sequence selected from SEQ ID NO: 3-5, 7-18, and 20-21. In some embodiments, the oligonucleotide comprises a nucleotide sequence selected from SEQ ID NO: 7, 9, 11, 12, 15-18, 20 and 21. In some embodiments, the oligonucleotide consists of a nucleotide sequence selected from SEQ ID NO: 7, 9, 11, 12, 15-18, 20 and 21. In some embodiments, the oligonucleotide does not substantially bind to HNRNPH1. In some embodiments, the oligonucleotide does not substantially inhibit translation of HNRNPH1. In some embodiments, the oligonucleotide does not substantially degrade HNRNPH1. In some embodiments, the oligonucleotide does not substantially inhibit expression of HNRNPH1. In some embodiments, inhibit is downregulate. In some embodiments, substantially is significantly. In some embodiments, the oligonucleotide comprises a nucleotide sequence selected from SEQ ID NO: 3-5, 7-10, 12-13, 15, 18, and 20-21. In some embodiments, the oligonucleotide consists of a nucleotide sequence selected from SEQ ID NO: 3-5, 7-10, 12-13, 15, 18, and 20-21. In some embodiments, the oligonucleotide comprises a nucleotide sequence selected from SEQ ID NO: 9, 15, 18, and 20-21. In some embodiments, the oligonucleotide consists of a nucleotide sequence selected from SEQ ID NO: 9, 15, 18, and 20-21. In some embodiments, the oligonucleotide comprises or consists of SEQ ID NO: 3. In some embodiments, the oligonucleotide comprises or consists of SEQ ID NO: 4. In some embodiments, the oligonucleotide comprises or consists of SEQ ID NO: 5. In some embodiments, the oligonucleotide comprises or consists of SEQ ID NO: 6. In some embodiments, the oligonucleotide comprises or consists of SEQ ID NO: 7. In some embodiments, the oligonucleotide comprises or consists of SEQ ID NO: 8. In some embodiments, the oligonucleotide comprises or consists of SEQ ID NO: 9. In some embodiments, the oligonucleotide comprises or consists of SEQ ID NO: 10. In some embodiments, the oligonucleotide comprises or consists of SEQ ID NO: 11. In some embodiments, the oligonucleotide comprises or consists of SEQ ID NO: 12. In some embodiments, the oligonucleotide comprises or consists of SEQ ID NO: 13. In some embodiments, the oligonucleotide comprises or consists of SEQ ID NO: 14. In some embodiments, the oligonucleotide comprises or consists of SEQ ID NO: 15. In some embodiments, the oligonucleotide comprises or consists of SEQ ID NO: 16. In some embodiments, the oligonucleotide comprises or consists of SEQ ID NO: 17. In some embodiments, the oligonucleotide comprises or consists of SEQ ID NO: 18. In some embodiments, the oligonucleotide comprises or consists of SEQ ID NO: 19. In some embodiments, the oligonucleotide comprises or consists of SEQ ID NO: 20. In some embodiments, the oligonucleotide comprises or consists of SEQ ID NO: 21. In some embodiments, the oligonucleotide comprises or consists of SEQ ID NO: 22.

In some embodiments, the oligonucleotide comprises a nucleotide sequence disclosed in Table 2. In some embodiments, the oligonucleotide comprises a nucleotide sequence selected from SEQ ID NO: 23-42. In some embodiments, the oligonucleotide comprises a nucleotide sequence selected from SEQ ID NO: 23-29, and 31-42. In some embodiments, the oligonucleotide consists of a nucleotide sequence selected from SEQ ID NO: 23-42. In some embodiments, the oligonucleotide consists of a nucleotide sequence selected from SEQ ID NO: 23-29, and 31-42. In some embodiments, the oligonucleotide comprises a nucleotide sequence selected from SEQ ID NO: 23-24, 26-27, 29-33 and 35-41. In some embodiments, the oligonucleotide consists of a nucleotide sequence selected from SEQ ID NO: 23-24, 26-27, 29-33 and 35-41. In some embodiments, the oligonucleotide comprises a nucleotide sequence selected from SEQ ID NO: 23-24, 26-27, 29, 31-33 and 35-41. In some embodiments, the oligonucleotide consists of a nucleotide sequence selected from SEQ ID NO: 23-24, 26-27, 29, 31-33 and 35-41. In some embodiments, the oligonucleotide comprises or consists of SEQ ID NO: 23. In some embodiments, the oligonucleotide comprises or consists of SEQ ID NO: 24. In some embodiments, the oligonucleotide comprises or consists of SEQ ID NO: 25. In some embodiments, the oligonucleotide comprises or consists of SEQ ID NO: 26. In some embodiments, the oligonucleotide comprises or consists of SEQ ID NO: 27. In some embodiments, the oligonucleotide comprises or consists of SEQ ID NO: 28. In some embodiments, the oligonucleotide comprises or consists of SEQ ID NO: 29. In some embodiments, the oligonucleotide comprises or consists of SEQ ID NO: 30. In some embodiments, the oligonucleotide does not comprises or consists of SEQ ID NO: 30. In some embodiments, the oligonucleotide comprises or consists of SEQ ID NO: 31. In some embodiments, the oligonucleotide comprises or consists of SEQ ID NO: 32. In some embodiments, the oligonucleotide comprises or consists of SEQ ID NO: 33. In some embodiments, the oligonucleotide comprises or consists of SEQ ID NO: 34. In some embodiments, the oligonucleotide comprises or consists of SEQ ID NO: 35. In some embodiments, the oligonucleotide comprises or consists of SEQ ID NO: 36. In some embodiments, the oligonucleotide comprises or consists of SEQ ID NO: 37. In some embodiments, the oligonucleotide comprises or consists of SEQ ID NO: 38. In some embodiments, the oligonucleotide comprises or consists of SEQ ID NO: 39. In some embodiments, the oligonucleotide comprises or consists of SEQ ID NO: 40. In some embodiments, the oligonucleotide comprises or consists of SEQ ID NO: 41. In some embodiments, the oligonucleotide comprises or consists of SEQ ID NO: 42.

In some embodiments, the oligonucleotide is not naturally occurring. In some embodiments, the oligonucleotide is man-made. In some embodiments, the oligonucleotide comprises at least one non-natural modification or structure. In some embodiments, the oligonucleotide comprises DNA and RNA. In some embodiments, the DNA and RNA are in the same strand. In some embodiments, the oligonucleotide is single stranded. In some embodiments, the oligonucleotide is a DNA/RNA chimera.

The term “nucleic acid” and “nucleotide” are well known in the art. A “nucleic acid” or “nucleotide” as used herein will generally refer to a molecule (i.e., a strand) of DNA, RNA, a combination thereof or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

In some embodiments, the oligonucleotide is chemically modified. In some embodiments, the chemical modification is a modification of a backbone of the oligonucleotide. In some embodiments, the chemical modification is a modification of a sugar of the oligonucleotide. In some embodiments, the chemical modification is a modification of a nucleobase of the oligonucleotide. In some embodiments, the chemical modification increases stability of the oligonucleotide in a cell. In some embodiments, the chemical modification increases stability of the oligonucleotide in vivo. In some embodiments, the chemical modification increases the oligonucleotide's ability to modulate translation. In some embodiments, the chemical modification increases the oligonucleotide's ability to induce degradation of a target mRNA. In some embodiments, the chemical modification increases the half-life of the oligonucleotide. In some embodiments, the chemical modification inhibits polymerase extension from the 3′ end of the oligonucleotide. In some embodiments, the chemical modification inhibits recognition of the oligonucleotide by a polymerase. In some embodiments, the chemical modification induces double-strand trigged degradation. In some embodiments, the chemically modified oligonucleotide triggers nucleic acid double-stranded degradation upon binding a target mRNA. In some embodiments, the chemical modification induces RISC-mediated degradation. In some embodiments, the chemical modification induces RISC-mediated degradation or any parallel nucleic acid degradation pathway.

In some embodiments, the oligonucleotide is devoid of a labeling moiety. In some embodiments, the oligonucleotide is not labeled. In some embodiments, the oligonucleotide does not emit a detectable signal or does not comprise moieties capable of being recognized so as to enable nucleic acid detection (e.g., digoxigenin and fluorescently labeled anti-DIG antibody). In some embodiments, a detectable signal comprises a dye or an emitting energy which provides detection of a compound, e.g., a polynucleotide, in vivo or in vitro. In some embodiments, a detectable signal comprises: a fluorescent signal, a chromatic signal, or a radioactive signal. In some embodiments, the oligonucleotide is devoid of radioactive nucleobase(s); digoxigenin, streptavidin, biotin, a fluorophore, hapten label, CLICK label, amine label, or thiol label.

In some embodiments, the oligonucleotide comprises a chemically modified backbone. In some embodiments, the chemically modified backbone is a backbone selected from: a 2-methoxyethyl (2′-MOE) backbone, a phosphate-ribose backbone, a phosphate-deoxyribose backbone, a phosphorothioate (PS) backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof. In some embodiments, a PS backbone is phosphorothioate-deoxyribose backbone. In some embodiments, a PS backbone is phosphorothioate-ribose backbone. In some embodiments, the backbone is a PS modified backbone. In some embodiments, the backbone is a 2′MOE backbone. In some embodiments, the entire backbone of the oligonucleotide is modified. In some embodiments, a region of the backbone of the oligonucleotide is modified. In some embodiments, all DNA bases of the backbone are modified. In some embodiments, all RNA bases of the backbone are modified. In some embodiments, all DNA and RNA bases comprise a PS backbone and all RNA bases comprise a 2′-MOE backbone. In some embodiments, a 2′-MOE backbone is a 2′-MOE modification.

In some embodiments, the oligonucleotide comprises at least one non-natural nucleotide. In some embodiments, a non-natural nucleotide is a non-canonical nucleotide. As used herein the terms “non-natural nucleotide” and “non-canonical nucleotide” refer to nucleic acid analogues. Non-limiting types of non-canonical nucleotide include Locked Nucleic Acid (LNA), peptide nucleic acid (PNA), modified DNA nucleotides, modified RNA nucleotides. Non-limiting examples of non-canonical RNA that were found to reduce the base pairing affinity include, 5-(Trifluoromethyl) uridine, 2 Methoxy-Adenine, 8-Nitro-Guanidine, 8-(trifluoromethyl)-Guanidine, 8-Carbo-methoxy-Guanidine, 5 Amino-Cytidine, and 5 Di Methyl-amino-Cytosine.

In some embodiments, the oligonucleotide comprises at least 14 bases, at least 15 bases, at least 16 bases, at least 17 bases, at least 18 bases, at least 19 bases, at least 20 bases, at least 21 bases, at least 22 bases, at least 23 bases, at least 24 bases, or at least 25 bases. Each possibility represents a separate embodiment of the invention. In some embodiments, the oligonucleotide comprises at least 18 bases. In some embodiments, the oligonucleotide comprises at least 20 bases.

In some embodiments, the oligonucleotide comprises at most 20 bases, at least 21 bases, at least 22 bases, at least 23 bases, at least 24 bases, at least 25 bases, at least 26 bases, at least 27 bases, at least 28 bases, at least 29 bases, at least 30 bases, or at least 35 bases. Each possibility represents a separate embodiment of the invention. In some embodiments, the oligonucleotide comprises at most 20 bases. In some embodiments, the oligonucleotide comprises at most 30 bases.

In some embodiments, the oligonucleotide comprises 12 to 30 bases, 12 to 29 bases, 12 to 28 bases, 12 to 27 bases, 12 to 26 based, 12 to 25 bases, 12 to 24 bases, 12 to 23 bases, 12 to 22 bases, 12 to 21 bases, 12 to 20 bases, 14 to 30 bases, 14 to 29 bases, 14 to 28 bases, 14 to 27 bases, 14 to 26 based, 14 to 25 bases, 14 to 24 bases, 14 to 23 bases, 14 to 22 bases, 14 to 21 bases, 14 to 20 bases, 15 to 30 bases, 15 to 29 bases, 15 to 28 bases, 15 to 27 bases, 15 to 26 based, 15 to 25 bases, 15 to 24 bases, 15 to 23 bases, 15 to 22 bases, 15 to 21 bases, 15 to 20 bases, 16 to 30 bases, 16 to 29 bases, 16 to 28 bases, 16 to 27 bases, 16 to 26 based, 16 to 25 bases, 16 to 24 bases, 16 to 23 bases, 16 to 22 bases, 16 to 21 bases, 16 to 20 bases, 17 to 30 bases, 17 to 29 bases, 17 to 28 bases, 17 to 27 bases, 17 to 26 based, 17 to 25 bases, 17 to 24 bases, 17 to 23 bases, 17 to 22 bases, 17 to 21 bases, 17 to 20 bases, 18 to 30 bases, 18 to 29 bases, 18 to 28 bases, 18 to 27 bases, 18 to 26 based, 18 to 25 bases, 18 to 24 bases, 18 to 23 bases, 18 to 22 bases, 18 to 21 bases, 18 to 20 bases, 19 to 30 bases, 19 to 29 bases, 19 to 28 bases, 19 to 27 bases, 19 to 26 based, 19 to 25 bases, 19 to 24 bases, 19 to 23 bases, 19 to 22 bases, 19 to 21 bases, 19 to 20 bases, 20 to 30 bases, 20 to 29 bases, 20 to 28 bases, 20 to 27 bases, 20 to 26 based, 20 to 25 bases, 20 to 24 bases, 20 to 23 bases, 20 to 22 bases, or 20 to 21 bases. Each possibility represents a separate embodiment of the invention. In some embodiments, the oligonucleotide comprises 17 to 22 bases. In some embodiments, the oligonucleotide comprises about 20 bases. In some embodiments, the oligonucleotide comprises 20 bases.

In some embodiments, the oligonucleotide is a GAPmer. In some embodiments, the oligonucleotide comprises DNA bases and RNA bases. In some embodiments, the oligonucleotide comprises a DNA core flanked by RNA. In some embodiments, the DNA core comprises at least 5, 6, 7, 8, 9 or 10 DNA bases. Each possibility represents a separate embodiment of the invention. In some embodiments, the DNA core comprises 10 DNA bases. In some embodiments, the DNA is flanked on the 5′ end by RNA. In some embodiments, the DNA is flanked on the 3′ end by RNA. In some embodiments, the oligonucleotide from 5′ to 3′ comprises RNA-DNA-RNA. In some embodiments, the two RNA flanks are of equal length. In some embodiments, the two RNA flanks are of different lengths. In some embodiments, an RNA flank comprises at least 1, 2, 3, 4, or 5 RNA bases. Each possibility represents a separate embodiment of the invention. In some embodiments, an RNA flank comprises 5 RNA bases. In some embodiments, the oligonucleotide comprises 5 RNA bases followed by 10 DNA bases followed by 5 RNA bases. In some embodiments, the 5′ end of the molecule is an RNA base. In some embodiments, the 3′ end of the molecule is an RNA base. In some embodiments, RNA is an RNA mimic. In some embodiments, an RNA base is an RNA mimic base. In some embodiments, binding of the DNA bases to the target mRNA induces RNase H mediated cleavage of the duplex. In some embodiments, binding of the DNA bases to the target mRNA induces RNase H mediated cleavage of the target mRNA. In some embodiments, RNase H mediated cleavage is RNase H mediated degradation. In some embodiments, the RNA ends increase affinity of the oligonucleotide to the mRNA. In some embodiments, the RNA ends increase resistance to nuclease mediated degradation.

In some embodiments, the oligonucleotide comprises at least 5, 6, 7, 8, 9, 10, 11, 12, 13, 14, 15, 16, 17, 18, 19 or 20 consecutive bases reverse complementary to the target mRNA. Each possibility represents a separate embodiment of the invention. In some embodiments, the consecutive bases are DNA bases. In some embodiments, the oligonucleotide comprises at least 5 consecutive bases. In some embodiments, the oligonucleotide comprises at least 10 consecutive bases. In some embodiments, the oligonucleotide comprises at least 12 consecutive bases. It will be understood by a skilled artisan that a stretch of DNA/RNA duplex is the target of RNase H and will induce cleavage and degradation of the target mRNA. In some embodiments, the oligonucleotide is an antisense oligonucleotide (ASO).

In some embodiments, the DNA bases comprise a backbone comprising a PS linkage. In some embodiments, the DNA bases comprise a backbone in which all the linkages are PS linkages. In some embodiments, the RNA bases comprise a backbone comprising a PS linkage. In some embodiments, the RNA bases comprise a backbone in which all the linkages are PS linkages. In some embodiments, all of the linkages in the backbone of the oligonucleotide comprise PS linkages. In some embodiments, the RNA bases comprise a 2′-MOE modification. In some embodiments, all of the RNA bases comprise 2′-MOE modifications. In some embodiments, all bases of the oligonucleotide comprise PS backbone and all RNA bases comprise a 2′-MOE modification.

In some embodiments, the 5′ flanking 5 RNA bases, 10 core DNA bases and 3′ flanking 5 RNA bases together comprise one of the sequences provided hereinabove. In some embodiments, the 5′ flanking 5 RNA bases, 10 core DNA bases and 3′ flanking 5 RNA bases together consist of one of the sequences provided hereinabove. In some embodiments, one of the sequences is a sequence selected from SEQ ID NO: 3-42. In some embodiments, the GAPmer comprises a chemically modified chimeric DNA/RNA molecules selected from those provided in SEQ ID NO: 43-82. In some embodiments, the GAPmer consists of a chemically modified chimeric DNA/RNA molecules selected from those provided in SEQ ID NO: 43-82. In some embodiments, the GAPmer comprises a chemically modified chimeric DNA/RNA molecules selected from those provided in SEQ ID NO: 43-62. In some embodiments, the GAPmer consists of a chemically modified chimeric DNA/RNA molecules selected from those provided in SEQ ID NO: 43-62. In some embodiments, the GAPmer a chemically modified chimeric DNA/RNA molecule selected from those provided in SEQ ID NO: 43-45, 47-58, and 60-61. In some embodiments, the GAPmer consists of a chemically modified chimeric DNA/RNA molecules selected from those provided in SEQ ID NO: 43-45, 47-58, and 60-61. In some embodiments, the GAPmer comprises a chemically modified chimeric DNA/RNA molecules selected from those provided in SEQ ID NO: 47, 49, 51, 52, 55-58, 60 and 61. In some embodiments, the GAPmer consists of a chemically modified chimeric DNA/RNA molecules selected from those provided in SEQ ID NO: 47, 49, 51, 52, 55-58, 60 and 61. In some embodiments, the GAPmer comprises a chemically modified chimeric DNA/RNA molecules selected from those provided in SEQ ID NO: 43-45, 47-50, 52-53, 55, 58, and 60-61. In some embodiments, the GAPmer consists of a chemically modified chimeric DNA/RNA molecules selected from those provided in SEQ ID NO: 43-45, 47-50, 52-53, 55, 58, and 60-61. In some embodiments, the GAPmer comprises a chemically modified chimeric DNA/RNA molecules selected from those provided in SEQ ID NO: 49, 55, 58, and 60-61. In some embodiments, the GAPmer consists of a chemically modified chimeric DNA/RNA molecules selected from those provided in SEQ ID NO: 49, 155, 58, and 60-61. In some embodiments, the GAPmer comprises a chemically modified chimeric DNA/RNA molecules selected from those provided in SEQ ID NO: 63-82. In some embodiments, the GAPmer comprises a chemically modified chimeric DNA/RNA molecules selected from those provided in SEQ ID NO: 63-69 and 71-82. In some embodiments, the GAPmer consists of a chemically modified chimeric DNA/RNA molecules selected from those provided in SEQ ID NO: 63-82. In some embodiments, the GAPmer consists of a chemically modified chimeric DNA/RNA molecules selected from those provided in SEQ ID NO: 63-69, and 71-82. In some embodiments, the GAPmer comprises a chemically modified chimeric DNA/RNA molecules selected from those provided in SEQ ID NO: 63-64, 66-67, 69-73 and 75-81. In some embodiments, the GAPmer consists of a chemically modified chimeric DNA/RNA molecules selected from those provided in SEQ ID NO: 63-64, 66-67, 69-73 and 75-81. In some embodiments, the GAPmer comprises a chemically modified chimeric DNA/RNA molecules selected from those provided in SEQ ID NO: 63-24, 66-67, 69, 71-73 and 75-81. In some embodiments, the GAPmer consists of a chemically modified chimeric DNA/RNA molecules selected from those provided in SEQ ID NO: 63-64, 66-67, 69, 71-73 and 75-81.

In some embodiments, the GAPmer comprises or consists of SEQ ID NO: 43. In some embodiments, the GAPmer comprises or consists of SEQ ID NO: 44. In some embodiments, the GAPmer comprises or consists of SEQ ID NO: 45. In some embodiments, the GAPmer comprises or consists of SEQ ID NO: 46. In some embodiments, the GAPmer comprises or consists of SEQ ID NO: 47. In some embodiments, the GAPmer comprises or consists of SEQ ID NO: 48. In some embodiments, the GAPmer comprises or consists of SEQ ID NO: 49. In some embodiments, the GAPmer comprises or consists of SEQ ID NO: 50. In some embodiments, the GAPmer comprises or consists of SEQ ID NO: 51. In some embodiments, the GAPmer comprises or consists of SEQ ID NO: 52. In some embodiments, the GAPmer comprises or consists of SEQ ID NO: 53. In some embodiments, the GAPmer comprises or consists of SEQ ID NO: 54. In some embodiments, the GAPmer comprises or consists of SEQ ID NO: 55. In some embodiments, the GAPmer comprises or consists of SEQ ID NO: 56. In some embodiments, the GAPmer comprises or consists of SEQ ID NO: 57. In some embodiments, the GAPmer comprises or consists of SEQ ID NO: 58. In some embodiments, the GAPmer comprises or consists of SEQ ID NO: 59. In some embodiments, the GAPmer comprises or consists of SEQ ID NO: 60. In some embodiments, the GAPmer comprises or consists of SEQ ID NO: 61. In some embodiments, the GAPmer comprises or consists of SEQ ID NO: 62. In some embodiments, the GAPmer comprises or consists of SEQ ID NO: 63. In some embodiments, the GAPmer comprises or consists of SEQ ID NO: 64. In some embodiments, the GAPmer comprises or consists of SEQ ID NO: 65. In some embodiments, the GAPmer comprises or consists of SEQ ID NO: 66. In some embodiments, the GAPmer comprises or consists of SEQ ID NO: 67. In some embodiments, the GAPmer comprises or consists of SEQ ID NO: 68. In some embodiments, the GAPmer comprises or consists of SEQ ID NO: 69. In some embodiments, the GAPmer comprises or consists of SEQ ID NO: 70. In some embodiments, the GAPmer does not comprises or consists of SEQ ID NO: 70. In some embodiments, the GAPmer comprises or consists of SEQ ID NO: 71. In some embodiments, the GAPmer comprises or consists of SEQ ID NO: 72. In some embodiments, the GAPmer comprises or consists of SEQ ID NO: 73. In some embodiments, the GAPmer comprises or consists of SEQ ID NO: 74. In some embodiments, the GAPmer comprises or consists of SEQ ID NO: 75. In some embodiments, the GAPmer comprises or consists of SEQ ID NO: 76. In some embodiments, the GAPmer comprises or consists of SEQ ID NO: 77. In some embodiments, the GAPmer comprises or consists of SEQ ID NO: 78. In some embodiments, the GAPmer comprises or consists of SEQ ID NO: 79. In some embodiments, the GAPmer comprises or consists of SEQ ID NO: 80. In some embodiments, the GAPmer comprises or consists of SEQ ID NO: 81. In some embodiments, the GAPmer comprises or consists of SEQ ID NO: 82.

By another aspect, there is provided a composition comprising a oligonucleotide of the invention.

In some embodiments, the composition is a pharmaceutical composition. In some embodiments, the composition comprises a pharmaceutically acceptable carrier excipient or adjuvant. In some embodiments, the composition comprises a therapeutically effective amount of the oligonucleotide of the invention.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

The dosage administered will be dependent upon the age, health, and weight of the recipient, kind of concurrent treatment, if any, frequency of treatment, and the nature of the effect desired. The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the pharmaceutical composition is formulated for administration. In some embodiments, administration is administration to a subject. In some embodiments, administration is systemic administration. In some embodiments, administration is local administration. In some embodiments, administration is administration to the brain. In some embodiments, the pharmaceutical composition is formulated to cross the blood brain barrier. In some embodiments, administration is cranial administration. In some embodiments, the pharmaceutical composition is formulated for performance of a method of the invention. In some embodiments, the pharmaceutical composition is for use in a method of the invention.

As used herein, the terms “administering,” “administration,” and like terms refer to any method which, in sound medical practice, delivers a composition containing an active agent to a subject in such a manner as to provide a therapeutic effect. One aspect of the present subject matter provides for intrathecal administration of a therapeutically effective amount of a composition of the present subject matter to a patient in need thereof. Other suitable routes of administration can include oral, parenteral, subcutaneous, intravenous, or intracranial.

The dosage administered will be dependent upon the age, health, and weight of the recipient, kind of concurrent treatment, if any, frequency of treatment, and the nature of the effect desired.

By another aspect, there is provided a method of treating or preventing an X-linked genetic disorder cause by a mutation in a gene on the X chromosome in a subject, the method comprising downregulating expression of the gene on the X chromosome in cells of the subject, thereby treating an X-linked genetic disorder.

In some embodiments, the method is a therapeutic method. In some embodiments, the method is a prophylactic method. In some embodiments, the genetic disorder is a single gene disorder. In some embodiments, the disorder is caused by a mutation in a single gene on the X chromosome. Simple and complex genetic disorders are well known in the art. A simple genetic disorder is one in which the cause is a genetic mutation in only one gene.

In some embodiments, the subject is a mammal. In some embodiments, the subject is a human. In some embodiments, the subject is male. In some embodiments, the subject is female. In some embodiments, the female subject is heterozygous for the mutation. In some embodiments, the subject is male and the subject is mosaic for the mutation. In some embodiments, the subject is in need of the method of the invention. In some embodiments, the subject is in need of treatment. In some embodiments, the subject suffers from the disorder. In some embodiments, the subject is determined to carry to the mutation. In some embodiments, at least one of the subject's parents carries the mutation. In some embodiments, the subject is male and the subjects mother carries the mutation. In some embodiments, the subject is female and at least one of the mother and father carriers the mutation.

In some embodiments, the disorder is a genetic disorder. In some embodiments, the cause of the disorder is a genetic mutation in the gen on the X chromosome. In some embodiments, the disorder is a developmental disorder. In some embodiments, the disorder is a neurological disorder. In some embodiments, the disorder is an epilepsy. In some embodiments, the disorder comprises epilepsy. In some embodiments, the disorder is characterized by epilepsy. In some embodiments, the disorder is a mental retardation. In some embodiments, the disorder comprises mental retardation. In some embodiments, the disorder is characterized by mental retardation. In some embodiments, the disorder is an autism spectrum disorder (ASD). In some embodiments, the disorder comprises autism or ASD. In some embodiments, the disorder is characterized by autism or ASD.

In some embodiments, the gene is HNRNPH2. In some embodiments, the disorder is an HNRNPH2-related developmental disorder. In some embodiments, the disorder is an HNRNPH2 caused developmental disorder. In some embodiments, the disorder is Mental retardation, X-linked, syndromic, Bain-type (MRXSB). MRXSB is also known as X-linked syndromic mental retardation, Bain type and Intellectual developmental disorder, X-linked, syndromic, Bain type and was first disclosed in Bain et al., 2016, “. Variants in HNRNPH2 on the X chromosome are associated with a neurodevelopmental disorder in females.” Am J Hum Genet; 99:728-734, herein incorporated by reference in its entirety.

In some embodiments, the gene is PCDH19. In some embodiments, the disorder is a PCDH19-related developmental disorder. In some embodiments, the disorder is a PCDH19 caused developmental disorder. In some embodiments, the disorder is Early Infantile Epileptic Encephalopathy type 9 (EIEE-9). This disorder is also known as Developmental and epileptic encephalopathy 9 (DEE9), and Early Infantile 9 and was first discovered in 1971 (see Juberg et al., 1971, “A new familial form of convulsive disorder and mental retardation limited to females.” J. Pediat. 79:726-732, herein incorporated by reference in its entirety. In some embodiments, the disorder is Epilepsy in Females with Mental Retardation (EFMR). EFMR is a rare early infantile epileptic encephalopathy (EIEE), phenotypically resembling Dravet syndrome (DS). In some embodiments, EIEE-9 is EFMR. In some embodiments, the disorder is DS. In some embodiments, the DS is SCN1A negative DS.

In some embodiments, the subject has been determined to have a mutation in the gene on the X-chromosome. In some embodiments, the method further comprises determining or confirming mutation of the gene on the X-chromosome. Genetic testing for somatic mutations is well known in the art and any such method can be employed. Standard testing methods comprise receiving a DNA sample from the subject and performing PCR or sequencing to detect genetic mutations.

In some embodiments, the mutation does not produce a dominant loss of function of in the protein encoded by the gene. In some embodiments, a dominant loss of function comprises rending a wild-type allele of the gene non-functional or with reduced function. In some embodiments, the mutation renders a cell-to-cell interaction non-functional or with reduced functionality. It will be understood by a skilled artisan that as the gene is on the X-chromosome any given cell will only produce either mutant or wild-type protein of the target gene. However, adjacent cells may have one cell that produces wild-type protein and one cell that produces mutant protein. The wild-type protein would be fully functional, however, its interaction with the mutant protein would be non-functional or have reduced functionality. This phenomenon would occur in heterozygous females and mosaic males. It may not occur in homozygous males, however. In some embodiments, the mutation does not cause the disorder is homozygous males. In some embodiments, homozygous males are non-mosaic homozygous males. In some embodiments, the subject comprises cells that express a wild-type allele of the gene and cells that express a mutant allele of the gene. In some embodiments, wild-type does not comprise the mutation. In some embodiments, a cell that expresses a wild-type allele expresses wild-type protein. In some embodiments, a cell that expresses a mutant allele expresses mutant protein.

In some embodiments, the downregulating expression is the cells that express the wild-type allele and the cells that express the mutant allele. In some embodiments, downregulation comprises downregulation of both mutated and unmutated copies of the gene. In some embodiments, downregulation comprises downregulation of both mutated and unmutated alleles of the gene. In some embodiments, downregulation comprises downregulation of both mutated and unmutated protein from the gene. In some embodiments, downregulation is inhibition.

In some embodiments, the disorder is neurological disorder and the cells are brain cells. In some embodiments, the downregulating expression is the brain cells. In some embodiments, administering is administering to the brain. In some embodiments, the subject comprises brain cells that express a wild-type allele of the gene and brain cells that express a mutant allele of the gene.

In some embodiments, downregulating comprises at least a 50, 55, 60, 65, 70, 75, 80, 85, 90, 92, 95, 97, 99 or 100% reduction in expression. Each possibility represents a separate embodiment of the invention. In some embodiments, downregulating comprises at least a 50% reduction. In some embodiments, downregulating comprises at least an 80% reduction. In some embodiments, downregulating comprises at least a 90% reduction. In some embodiments, expression is mRNA expression. In some embodiments, expression is protein expression. Methods of measuring mRNA and protein expression are well known in the art and any such method may be used to test/confirm expression levels. These methods include but are not limited to RT-PCR, qPCR, microarrays, sequencing, immunostaining, immunoblotting, protein arrays and many others known to the skilled artisan.

In some embodiments, the method comprises administering an oligonucleotide reverse complementary to an mRNA of the gene. In some embodiments, the oligonucleotide comprises 12 to 30 bases, 12 to 29 bases, 12 to 28 bases, 12 to 27 bases, 12 to 26 based, 12 to 25 bases, 12 to 24 bases, 12 to 23 bases, 12 to 22 bases, 12 to 21 bases, 12 to 20 bases, 14 to 30 bases, 14 to 29 bases, 14 to 28 bases, 14 to 27 bases, 14 to 26 based, 14 to 25 bases, 14 to 24 bases, 14 to 23 bases, 14 to 22 bases, 14 to 21 bases, 14 to 20 bases, 15 to 30 bases, 15 to 29 bases, 15 to 28 bases, 15 to 27 bases, 15 to 26 based, 15 to 25 bases, 15 to 24 bases, 15 to 23 bases, 15 to 22 bases, 15 to 21 bases, 15 to 20 bases, 16 to 30 bases, 16 to 29 bases, 16 to 28 bases, 16 to 27 bases, 16 to 26 based, 16 to 25 bases, 16 to 24 bases, 16 to 23 bases, 16 to 22 bases, 16 to 21 bases, 16 to 20 bases, 17 to 30 bases, 17 to 29 bases, 17 to 28 bases, 17 to 27 bases, 17 to 26 based, 17 to 25 bases, 17 to 24 bases, 17 to 23 bases, 17 to 22 bases, 17 to 21 bases, 17 to 20 bases, 18 to 30 bases, 18 to 29 bases, 18 to 28 bases, 18 to 27 bases, 18 to 26 based, 18 to 25 bases, 18 to 24 bases, 18 to 23 bases, 18 to 22 bases, 18 to 21 bases, 18 to 20 bases, 19 to 30 bases, 19 to 29 bases, 19 to 28 bases, 19 to 27 bases, 19 to 26 based, 19 to 25 bases, 19 to 24 bases, 19 to 23 bases, 19 to 22 bases, 19 to 21 bases, 19 to 20 bases, 20 to 30 bases, 20 to 29 bases, 20 to 28 bases, 20 to 27 bases, 20 to 26 based, 20 to 25 bases, 20 to 24 bases, 20 to 23 bases, 20 to 22 bases, or 20 to 21 bases reverse complementary to an mRNA of the gene. Each possibility represents a separate embodiment of the invention. In some embodiments, the oligonucleotide comprises 12 to 30 bases reverse complementary to an mRNA of the gene. In some embodiments, the oligonucleotide comprises 16 to 24 bases reverse complementary to an mRNA of the gene. In some embodiments, the oligonucleotide comprises 18 to 22 bases reverse complementary to an mRNA of the gene. In some embodiments, the oligonucleotide comprises about 20 bases reverse complementary to an mRNA of the gene.

In some embodiments, the oligonucleotide is an oligonucleotide of the invention. In some embodiments, the administering is administering a pharmaceutical composition comprising the oligonucleotide. In some embodiments, the pharmaceutical composition is a pharmaceutical composition of the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

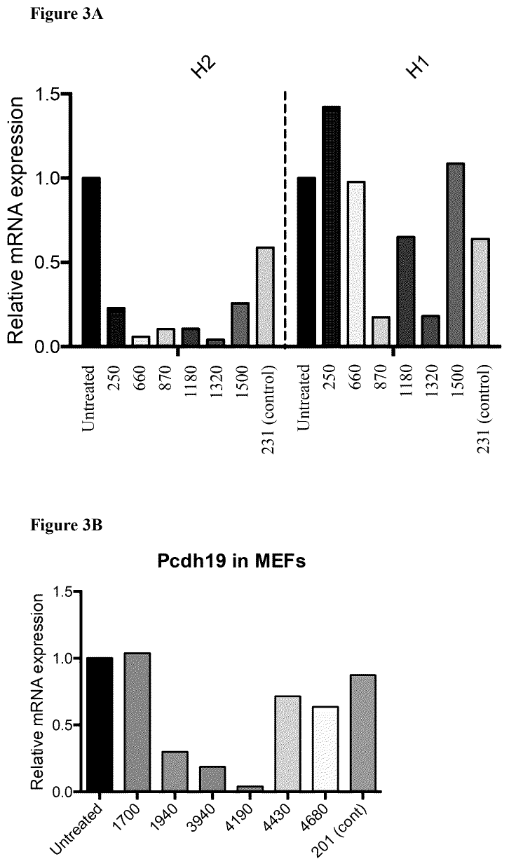

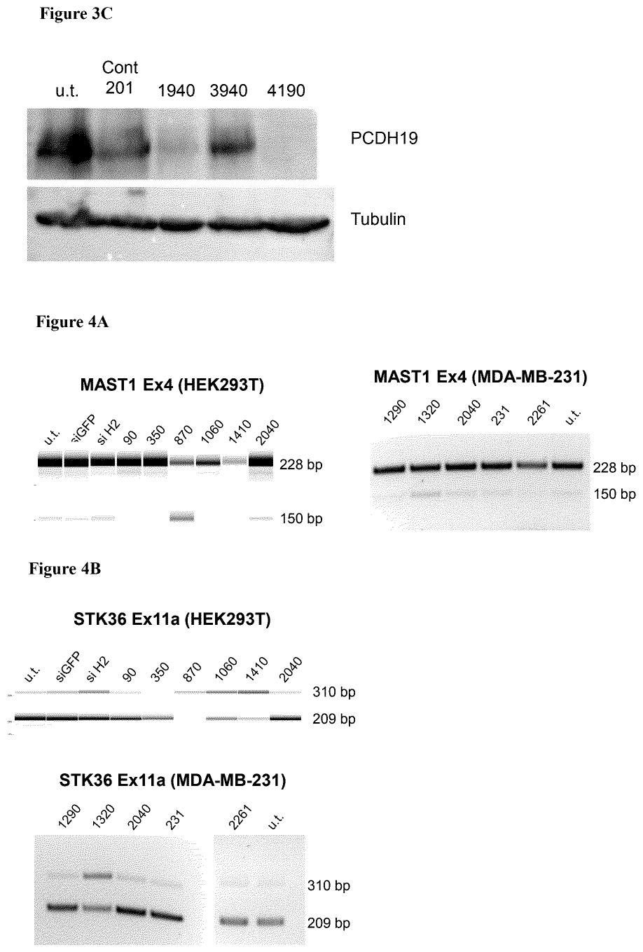

Cell cultures and GAPmer transfections. All cell lines were grown in DMEM supplemented with 10% FBS and 1% Penicillin/Streptomycin. Cells were grown in a 5% CO₂ incubator at 37° C. Transfection of GAPmers into cells was performed on cells at 50-70% confluency, that were seeded the day before. Cells were transfected using the Calcium Phosphate method with each GAPmer at 50 nM final concentration. Media was replaced on the following day and cells were harvested on the next day (48 hours post transfection). For the 24 hours transfection (FIG. 3A), cells were harvested 24 hours after transfection without media replacement.

GAPmer sequences and chemistry. The GAPmers are 20 bases long and are made of a 10 DNA bases core and 5 RNA bases on each side, flanking the DNA. All linkages throughout the backbone (both RNA and DNA) are phosphorothioate (PS). GAPmer sequences are provided in Table 1 and 2. All RNA based are 2′-MOE modified. Bracketed GAPmers did not work well in downregulating the gene or worked only in one human cell line out of two that were tested.

TABLE 1
HNRNPH2 GAPmer sequences.

| Name | Sequence (5′-3′) (SEQ ID NO:) |
|---|---|
| 40 | TAGACGACGACCAGAACTAC (3) |
| 90 | ATACCGGGTAATTTTCCTCC (4) |
| 250 | ATCAGAGAAGAAGCGCATCA (5) |
| 350 | [CTTCCTCTTCAGATTCAAGT] (6) |
| 450 | GTATGCTTCAACACCCAATC (7) |
| 540 | AACTGAACAATCTCTTCCTT (8) |
| 660 | TTTAAGGCCTTCTCAGCTAT (9) |
| 750 | TTTCGAGGGGGATCATAGTG (10) |
| 870 | TACCCTCCACCATAGGCACC (11) |
| 950 | AGTAATTGAGGTCTCTTCCA (12) |
| 1060 | ATTCTCAGTGGCTCTGTAAG (13) |
| 1180 | TGCCACAGCATCTTCATGAG (14) |
| 1290 | AAAAGTTCTACATAGCTGTG (15) |
| 1320 | CCACCACTTGCCCCTGCTGT (16) |
| 1410 | TAACCACCACTCAGCTGCTG (17) |
| 1500 | TAAGCAAGGTTTGACTGATA (18) |
| 1680 | [TTCTTAAAAAAGGAGAAAGA] (19) |
| 1870 | AACTAACTTGAGGAAAGCTA (20) |
| 2040 | TTCACTTTGAATCGTAGACA (21) |
| 2280 | [AAAGGAAGATTTATTTAACA] (22) |

“Name” corresponds to the position in SEQ ID NO: 1 at which hybridization occurs.
Thus, Gapmer #40 is reverse-complementary to nucleotides 40-21 of SEQ ID NO: 1.

TABLE 2
PCDH19 GAPmer sequences:

| Name | Sequence (5′-3′) (SEQ ID NO:) |
|---|---|
| 220 | GGTTTAGGATTTCGGATGAA (23) |
| 1030 | GGGAGTCAGTTACTGGCAAG (24) |
| 1700 | [CACCGGCAGCAGGAGCGACT] (25) |
| 1940 | GTCAATCTTCTGCTTGGTGA (26) |
| 2190 | CGTTGGGCGTGAGCTCGTAA (27) |
| 2440 | [GTGTTGGGAGGCGAGTTTTC] (28) |
| 2690 | GTCCAGCACGCTGACGGTGA (29) |
| 2940 | TGAGGTTGTATTGGTCGTGC (30) |
| 3200 | GATGGAGACATAGGTGAAGA (31) |
| 3440 | AGTCACCAGGTAGCCTATGC (32) |
| 3690 | TTGACTCTTGGGCATCGAGA (33) |
| 3940 | [TCCTCTGTCTTTTTGTCTTG] (34) |
| 4190 | CTGGTTCTCCACATTCAGGA (35) |
| 4430 | CTCACTGTCAGTTTGGTCAC (36) |
| 4680 | CATCAGCAGCAGTAGCTTCA (37) |
| 4940 | AGCCAGGGCAATGGTGTAAG (38) |
| 5240 | GGAAAAGCTCTGAAATATAG (39) |
| 6260 | CAGCCCATGACGTGCTTTGC (40) |
| 7310 | ATAGGCAGCCATCATAACTT (41) |
| 8420 | [TATCCCCTTCACCCTGCCTC] (42) |

RNA preparation and quantitative real-time PCR (qRT-PCR). Cells were collected in TRI-reagent (Sigma) and total RNA was extracted using standard procedures. RNA was reverse transcribed using the iScript cDNA Synthesis kit (Bio-Rad) and quantitative RT-PCR was performed using iTaq Universal SYBR Green Supermix (Bio-Rad), with primers that were designed using Primer3 tool (Table 3). The qRT-PCR was run on the 96-well plate CFX Connect (Bio-Rad) and analyzed with the Bio-Rad CFX Maestro software.

Protein preparation and Western blotting. Cells were lysed in RIPA lysis buffer for whole protein preparation. Protein concentrations were measured using the DC Protein Assay kit (Bio-Rad) on a plate reader, at 750 nm wavelength. 40 μg of protein was loaded and run on an SDS page using the Mini-PROTEAN Gel Electrophoresis system. Proteins were transferred onto a PVDF membrane (Merck Millipore) using Trans-Blot Turbo transfer system (Bio-Rad) and blocked with 5% milk in TBST. Blots were incubated with primary antibodies at 4° C. overnight, washed with TBST and incubated with secondary antibodies. Detection was performed with ECL on a Gel Doc XR+ imaging system (Bio-Rad).

Reverse Transcriptase PCR (RT-PCR). RNA was reverse transcribed using the iScript cDNA Synthesis kit (Bio-Rad). PCR was performed with PCRBIO HS Taq Mix Red (PCR Biosystems) using an annealing temperature of 60° C. Primers (Table 3) were designed to detect specific exons that are subjected to alternative splicing. PCR products were either separated on a 2% Agarose gel or run on the LabChip GX Touch HT (PerkinElmer).

TABLE 3
Primers for RT-PCR

| | Forward Primer | Reverse Primer |
|---|---|---|
| Gene | (5′-3′) (SEQ ID NO:) | (5′-3′) (SEQ ID NO:) |
| MAST1 | CACTTCTCGTTTGCCTCCTC | CACCATCCTCGTCTGTGATG |
| | (84) | (85) |
| STK36 | TGCCTATTCAACTGAAGGCT | GGCAGAAGGAATACAAGGCA |
| | CC (86) | AC (87) |
| MADD | CAACAGCTTGAGACTGGCAA | CTAGAATTGCTGGATCGACC |
| | GTGA (88) | CTGGG (89) |
| LSM14B | GAGAAGGGGGAAGAGAAGGA | TCAGTTATGCGCCACTTCAG |
| | (90) | (91) |
| SAT1 | TTTGGAGAGCACCCCTTTTA | CAATCCACGGGTCATAGGTA |
| | (92) | A (93) |
| ACTIN | CGTGGACATCCGCAAAG | GGAAGGTGGACAGCGAG |
| | (94) | (95) |

Intracerebroventricular (ICV) injection. HNRNPH2-targeting GAPmers were injected via stereotactic surgery into the ventricle of NOD-SCID mice, 0.1 mm posterior and 1 mm lateral to the bregma, at a depth of 2.5 mm. The GAPmers were dissolved in PBS and injected at a 3.6 μl volume containing either 100 μg (n=3), 50 μg (n=3) or 25 μg (n=3) of the GAPmer. PBS (vehicle) was injected as a control (n=3). For PCDH19-targeting GAPmer injection, a cannula was installed on the skull via stereotactic surgery and placed in the ventricle of C57BL/6 mice, 0.1 mm posterior and 1 mm lateral to the bregma, at a depth of 2.5 mm. A week later, 50 μg of PCDH19 GAPmer (4190) was injected through the cannula at 2.2 μl volume, 3 times a week for two weeks. As control, mice were injected with a sense GAPmer (8401) or PBS. 10 mice were used per group. Mice were sacrificed 14 days after the first GAPmer injection and brain tissue was homogenized in TRI-reagent for RNA analysis.

### Example 1: Antisense Oligonucleotide GAPmer Screen Identifies Sequences that Target HNRNPH2 or PCDH19

20 sequences along the HNRNPH2 transcript (Table 1) were selected for GAPmer generation (SEQ ID NO: 43-62) and tested for their efficiency in downregulating HNRNPH2 levels. Similarly, 20 sequences along the PCDH19 transcript (Table 2) were selected for GAPmer generation (SEQ ID NO: 63-82) and tested for PCDH19 downregulation. All 20 GAPmers of each gene were transfected individually into two different human cell lines, HEK293T and OVCA433, to identify the optimal sequence(s) for depletion.

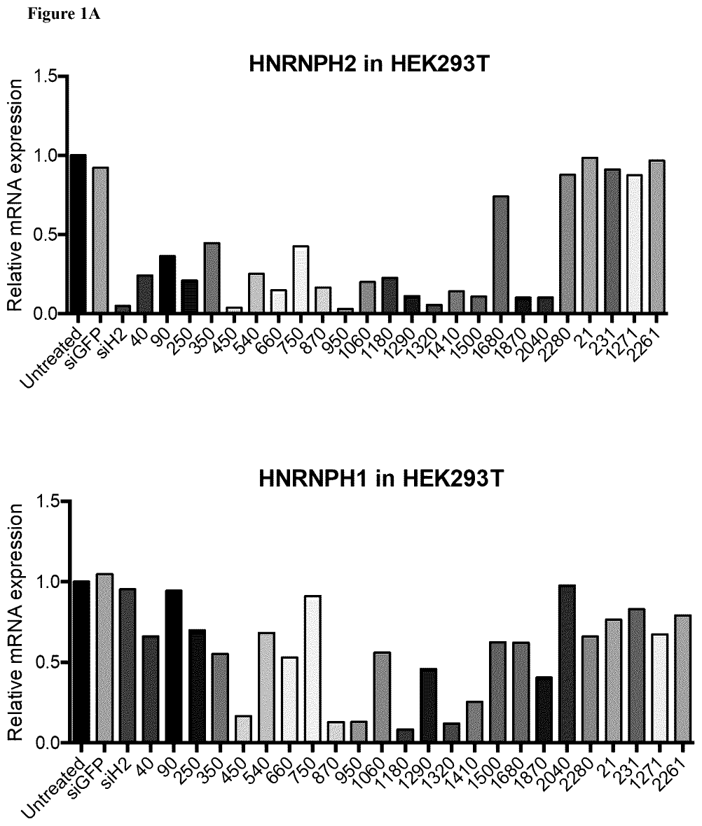

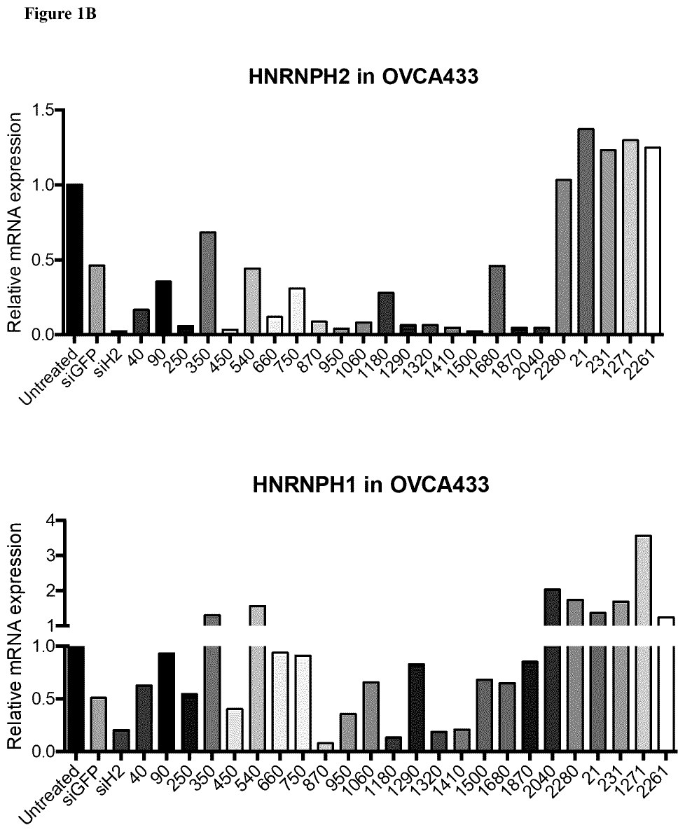

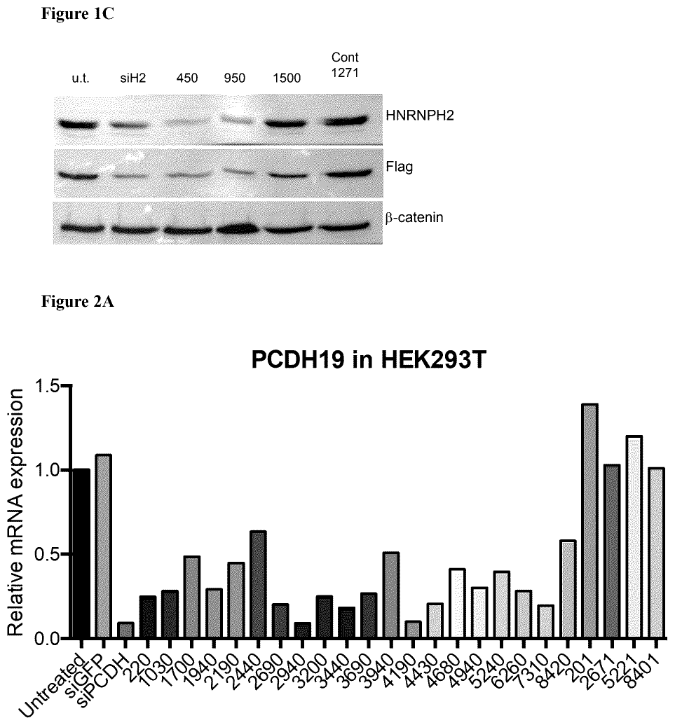

Quantitative real-time PCR (qRT-PCR) demonstrated that 18 out of the 20 HNRNPH2 GAPmers reduced HNRNPH2 transcript at various efficiencies (SEQ ID NO: 43-58, and 60-61). 10 GAPmers reduced HNRNPH2 levels by at least 80% in both cell lines (SEQ ID NO: 47, 49, 51, 52, 55-58, 60 and 61). From these, 5 GAPmers were specific to HNRNPH2 (660, 1290, 1500, 1870 and 2040; SEQ ID NO: 49, 55, 58, and 60-61), while the other 5 also reduced the levels of the close family member HNRNPH1 (450, 870, 950, 1320 and 1410; SEQ ID NO: 47, 51-52, and 56-57) (FIG. 1A-1B). Transfection of some of these GAPmers into Lan2 neuroblastoma cells resulted in decreased protein levels of the stably expressed exogenous Flag-HNRNPH2 (FIG. 1C).

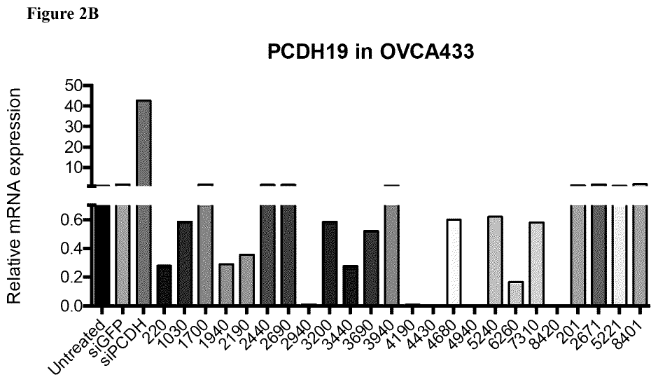

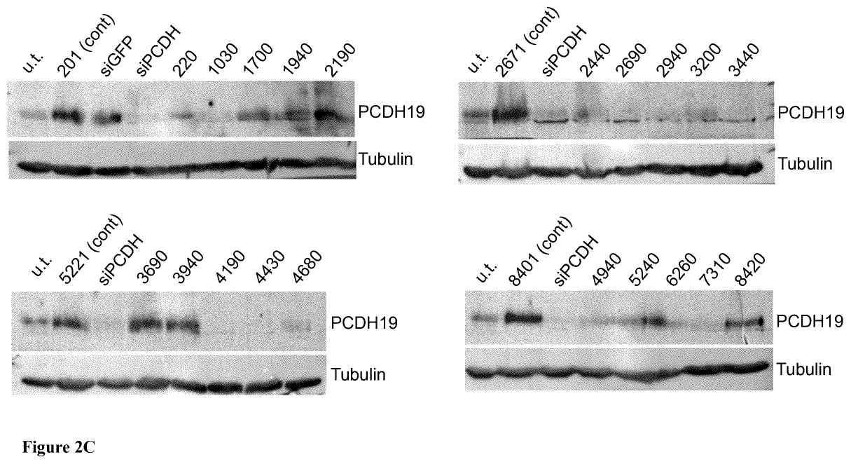

Of the 20 PCDH19 GAPmers, 18 reduced gene expression at various efficiencies. However, only 3 GAPmers (2940, 4190 and 4430; SEQ ID NO: 70, 75, 76) reduced PCDH19 levels by more than 80% in the two cell lines (FIG. 2A-2B). Protein levels of PCDH19 were drastically decreased using these GAPmers in OVCA433 cells (FIG. 2C).

Some of the GAPmer sequences were identical or almost identical between the human and the mouse sequence. To assess the usefulness of these GAPmers for gene knockdown in mouse tissue, we transfected them into mouse cell lines and measured gene expression. The 6 GAPmers of HNRNPH2 that were tested reduced murine Hnrnph2 by 80% or more (FIG. 3A). Two of those GAPmers also reduced Hnrpnph1 as expected from their sequence identity (FIG. 3A). Of the 6 PCDH19 GAPmers that were tested in mouse cells, only two reduced murine Pcdh 19 levels by at least 80% (FIG. 3B).

### Example 2: GAPmers Targeting Both HNRNPH2 and HNRNPH1 Affect Splicing

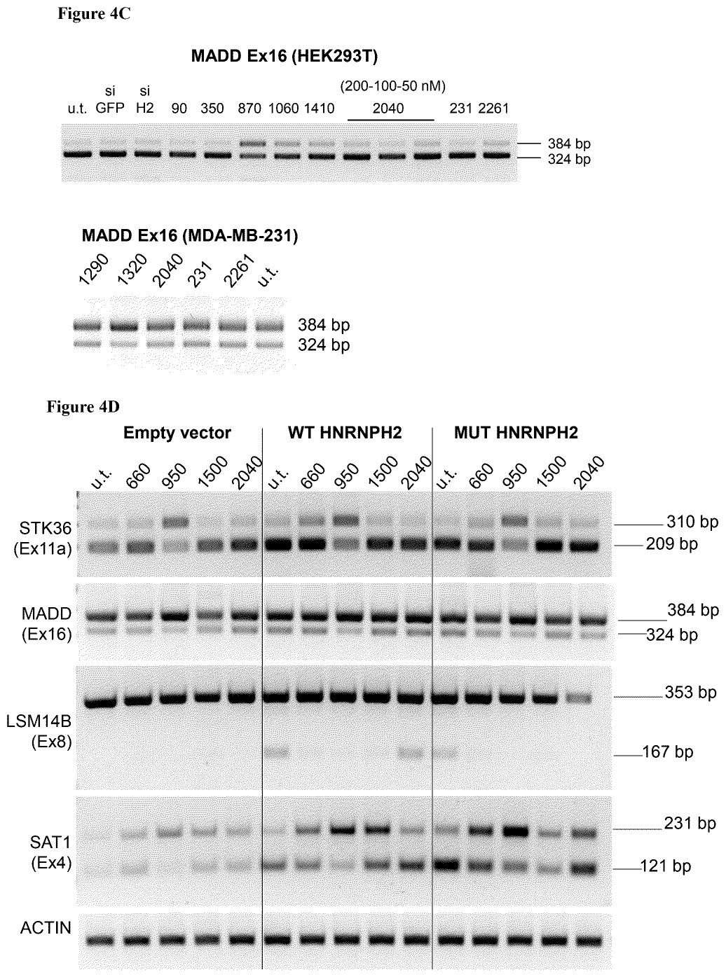

hnRNP family members play pleiotropic roles in RNA metabolism and regulation processes, such as alternative splicing. However, splicing events that are specifically mediated by hnRNPH2 are still unknown, nor are their association with the neurodevelopmental pathology. We performed reverse transcription PCR (RT-PCR) for genes that are established splicing targets of hnRNPA and hnRNPH1, in order to examine whether they are also affected by hnRNPH2. Cells treated with GAPmer that reduces both HNRNPH2 and HNRNPH1 showed splicing changes in three of the tested genes: Microtubule Associated Serine/Threonine Kinase 1 (MAST1), Serine/Threonine Kinase 36 (STK36) and MAP Kinase Activating Death Domain (MADD). Dual depletion of HNRNPH2 and HNRNPH1 (GAPmer 870 in HEK293T or 1320 in MDA-MB-231) resulted in skipping of exon 4 of MAST1 (FIG. 4A), inclusion of exons 11-12 of STK36 (FIG. 4B) and inclusion of exon 16 of MADD (the IG20 isoform) (FIG. 4C).

To find splicing events that are HNRNPH2-specific, we performed deep RNA sequencing on MDA-MB-231 cells transfected with GAPmers that are specific for HNRNPH2, as well as on cells that ectopically express wild-type (WT) or mutant (MUT) HNRNPH2. Results demonstrate that HNRNPH2-targeting GAPmers rescue splicing alterations caused by ectopic HNRNPH2 expression. STK36 and MADD show splicing changes only when the cells were treated with GAPmer 950, which downregulates both HNRNPH2 and HNRNPH1. However, the splicing patterns of LSM14B and SATI, two candidates from the RNA sequencing analysis, also change upon treatment with GAPmers that are specific for HNRNPH2 (FIG. 4D).

### Example 3: Injection of GAPmer into the Mouse Brain Reduces HNRNPH2 Levels

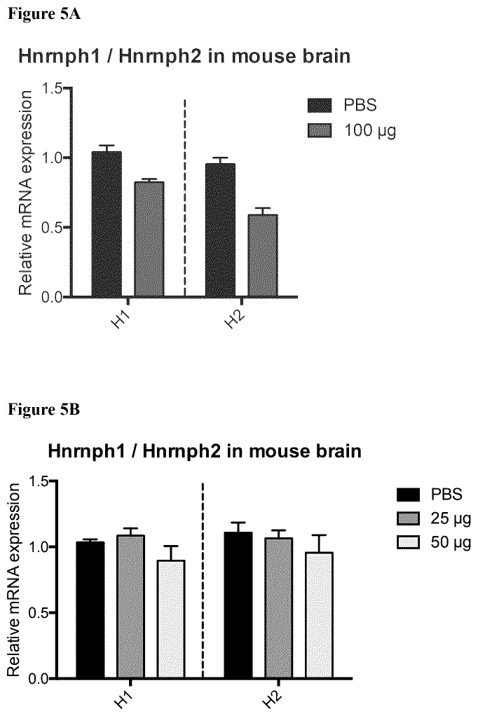

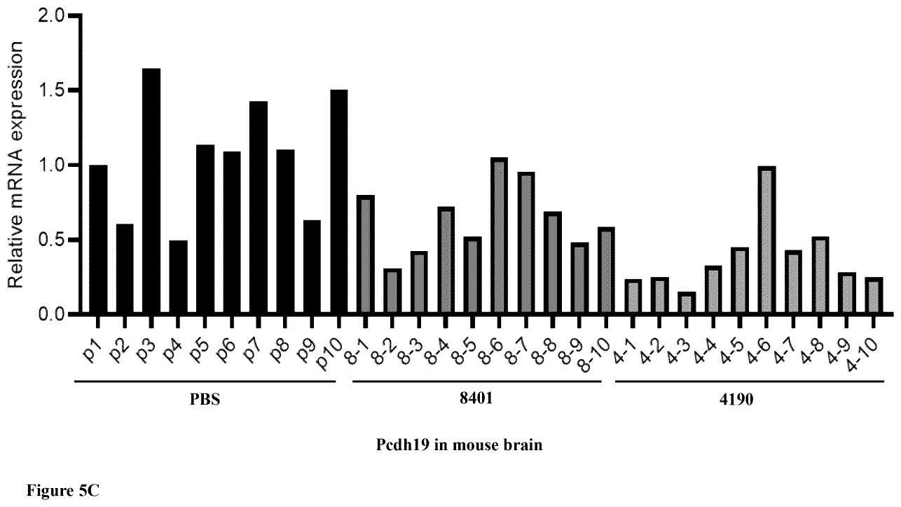

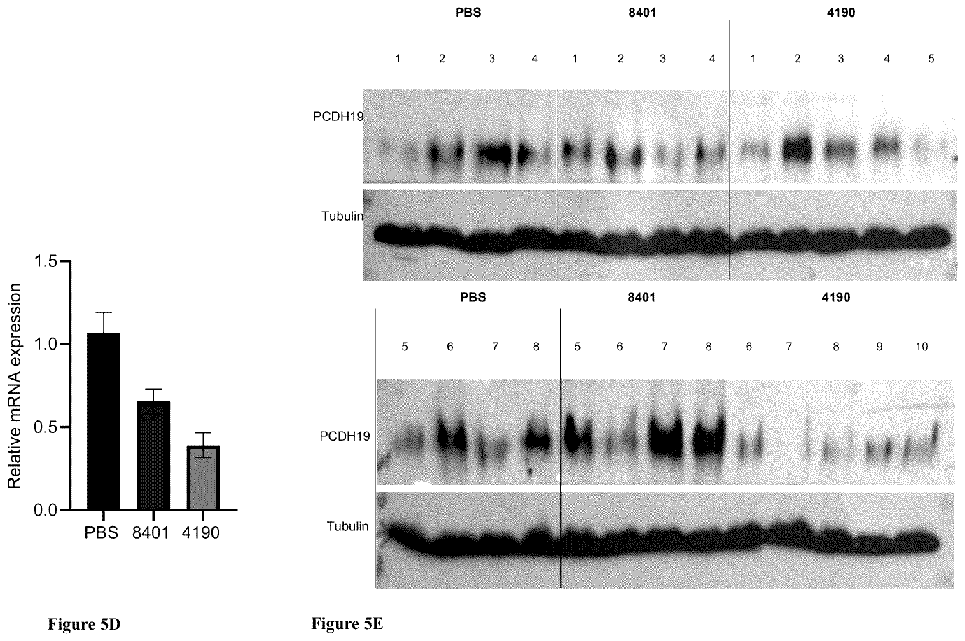

The ultimate goal of the project is to silence HNRNPH2 and PCDH19 in the brain tissue, in order to alleviate disease phenotypes. To test the efficacy of the GAPmers in vivo, we injected HNRNPH2-specific GAPmer into the brain of wild-type mice, by intracerebroventricular (ICV) injection. The GAPmer was injected in varying concentrations and the mice were sacrificed at different time points after the injection. Mice that were injected with 100 μg of HNRNPH2-specific GAPmer showed 40% downregulation of the Hnrnph2 transcript in their brains 14 days after the injection (FIG. 5A). Lower doses (50 or 25 μg) did not affect Hnrnph2 expression (FIG. 5B). A GAPmer targeting PCDH19 (4190) was tested by repeated injections into the brain through a cannula we installed in the skull. We injected 50 μg of GAPmer 3 times a week for two weeks, for a total of 300 μg, and sacrificed the mice 14 days after the first injection (FIG. 5C). We observed an ˜60% decrease in Pcdh19 expression in the PCDH19 GAPmer group compared to PBS-injected mice (FIG. 5D), however the variation within the group was high. Western blotting analysis of individual mice showed that 7 mice out of 10 had significantly decreased PCDH19 protein levels in the brain, attesting to the efficiency of this GAPmer in downregulating PCDH19 (FIG. 5E). Validated GAPmers are evaluated for their capacity to reverse the disease phenotypes of mouse models that recapitulate the neurodevelopmental diseases.

GAPmers are tested in vitro in a model of induced neurons that are differentiated from patient-derived skin fibroblasts (which share the X-linked mutation). Electrophysiological assays such as electroencephalogram (EEG) are performed on the induced neurons from patients and compared to neurons differentiated from healthy individuals. The ability of GAPmer treatment to reverse mutant neuron disfunction into normal activity is evaluated.

Mouse models for MRXSB and EIEE-9 were generated. For HNRNPH2, two point mutations were inserted to convert Arginine 206 to Tryptophan (R206W). For PCDH19, Tyrosine 516 was converted into a stop codon (Y516X). GAPmers are tested in vivo for their capacity to reverse disease phenotype by injection into brains of HNRNPH2- and PCDH19-mutant mice. Mice are tested in various muscle strength and behavioral assays to evaluate reversal of the neurological phenotype.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. (canceled)

2. (canceled)

3. (canceled)

4. (canceled)

5. (canceled)

6. (canceled)

7. A method of treating a neurodevelopmental disorder caused by a mutation in protocadherin 19 (PCDH19) in a subject in need thereof, the method comprising administering to the brain of said subject an antisense oligonucleotide comprising or consisting of a sequence selected from SEQ ID NO: 23-24, 26-27, 29, 31-33, and 35-41.

8. (canceled)

9. The method of claim 7, wherein said neurodevelopmental disorder is selected from Early Infantile Epileptic Encephalopathy type 9 (EIEE-9) and Epilepsy in Females with Mental Retardation (EFMR).

10. The method of claim 7, wherein said neurodevelopmental disorder is characterized by epilepsy, autism spectrum disorder (ASD), or both.

11. The method of claim 7, wherein said mutation does not produce a dominant loss of function that renders a wild-type allele of said single gene non-functional or with a reduced function.

12. The method of claim 7, wherein said subject comprises brain cells that express a wild-type allele of said single gene and brain cells that express a mutant allele of said single gene.

13. (canceled)

14. (canceled)

15. The method of claim 7, wherein said antisense oligonucleotide comprises between 12 and 30 bases.

16. The method of claim 15, wherein said antisense oligonucleotide comprises a chemically modified backbone, at least one non-natural nucleotide, both DNA and RNA bases or a combination thereof.

17. The method of claim 16, wherein said antisense oligonucleotide comprises a chemically modified backbone comprising phosphorothioate (PS) linkages.

18. The method of claim 15, wherein said antisense oligonucleotide comprises a DNA core flanked both 5′ and 3′ by RNA bases, optionally comprising 10 DNA bases flanked by 5 RNA bases 5′ and 5 RNA bases 3′.

19. The method of claim 18, wherein said RNA bases comprise a 2′-MOE modification.

20. (canceled)

21. The method of claim 15, wherein said antisense oligonucleotide is a GAPmer.

22. The method of claim 15, wherein said antisense oligonucleotide comprises at least 5 consecutive DNA bases reverse complementary to an mRNA of PCDH19, such that hybridization of said DNA bases to said mRNA induces RNase H mediated cleavage and degradation of said mRNA.

23. The method of claim 15, wherein said antisense oligonucleotide is specific to PCDH19 and does not substantially bind to an mRNA of any other gene.

24. (canceled)

25. (canceled)

26. (canceled)

27. An antisense oligonucleotide capable of binding to an mRNA of PCDH19 and inducing degradation of said mRNA and comprising a nucleotide sequence selected from SEQ ID NO: 23-24, 26-27, 29, 31-33 and 35-41.

28. The antisense oligonucleotide of claim 27, wherein said antisense oligonucleotide comprises a chemically modified backbone, at least one non-natural nucleotide, both DNA and RNA bases or a combination thereof.

29. The antisense oligonucleotide of claim 28, wherein said antisense oligonucleotide comprises a chemically modified backbone comprising phosphorothioate (PS) linkages.

30. The antisense oligonucleotide of claim 27, wherein said antisense oligonucleotide comprises a DNA core flanked both 5′ and 3′ by RNA bases, optionally comprising 10 DNA bases flanked by 5 RNA bases 5′ and 5 RNA bases 3′.

31. The antisense oligonucleotide of claim 30, wherein said RNA bases comprise a 2′-MOE modification.

32. (canceled)

33. The antisense oligonucleotide of claim 27, wherein said antisense oligonucleotide is a GAPmer.

34. (canceled)

35. A pharmaceutical composition comprising an antisense oligonucleotide of claim 27 and a pharmaceutically acceptable carrier excipient or adjuvant.

36. (canceled)


SEQUENCE LISTING (95 sequences)

SEQ ID NO: 1 - DNA, 2291 bp, Homo sapiens
  source 1..2291: mol_type = other DNA
actcgcttacgtgagcagaagtagttctggtcgtcgtctaccgtctcgctatagccgtttgagggaagaaggaggaaaattacccggtatcgttagagctacaccaaaattgcattgagccaaacttgccaccaagagcccaacaatcaccatgatgctgagcacggaaggcagggaggggttcgtggtgaaggtcaggggcctaccctggtcctgctcagccgatgaagtgatgcgcttcttctctgattgcaagatccaaaatggcacatcaggtattcgtttcatctacaccagagaaggcagaccaagtggtgaagcatttgttgaacttgaatctgaagaggaagtgaaattggctttgaagaaggacagagaaaccatgggacacagatacgttgaagtattcaagtctaacagtgttgaaatggattgggtgttgaagcatacaggtccgaatagccctgatactgccaacgatggcttcgtccggcttagaggactcccatttggctgtagcaaggaagagattgttcagttcttttcagggttggaaattgtgccaaatgggatgacactgccagtggactttcaggggcgaagcacaggggaagcctttgtgcagtttgcttcacaggagatagctgagaaggccttaaagaaacacaaggaaagaatagggcacaggtacattgagatcttcaagagtagccgagctgaagttcgaacccactatgatccccctcgaaagctcatggctatgcagcggccaggtccctatgataggccgggggctggcagagggtataatagcattggcagaggagctgggtttgaaaggatgaggcgtggtgcctatggtggagggtatggaggctatgatgactatggtggctataatgatggatatggctttgggtctgatagatttggaagagacctcaattactgtttttcaggaatgtctgatcatagatacggagatggtgggtccagtttccagagcaccacagggcactgtgtacacatgagggggttaccttacagagccactgagaatgatatttataatttcttctcacctcttaatcccatgagagtacatattgaaattggacccgatggcagagttaccggtgaggcagatgttgaatttgctactcatgaagatgctgtggcagctatggcaaaagacaaagctaatatgcaacacagatatgtggagctcttcttaaattctactgcaggaacaagtgggggtgcttacgatcacagctatgtagaactttttttgaattctacagcaggggcaagtggtggcgcttatggtagccaaatgatgggagggatgggcttatccaaccagtctagttatggaggtcctgctagccagcagctgagtggtggttatggaggtggttatggtggtcagagcagtatgagtggatatgaccaagttctgcaggaaaactccagtgactatcagtcaaaccttgcttaggtagagaaggagcactaaatagctactccagatataaaagctgtacatttgtgggagttgaatagaatgggagggatgtttagtatatccagtatgattggtaaatgggaaatataattgattctgatcactcttggtcagcttctctttctttatctttctttctccttttttaagaaaacgagttaagtttaacagttttgcattacaggcttgtgattcatgcttactgtaaagtggaagttgagattattttaaaacttcaagctcagtaattttgaacactgaaacattcatctaggacataataacaaagttcagtattgaccataactgttaaaacaatttttagctttcctcaagttagttatgttgtaggagtgtacctaagcagtaagcgtatttaggttaatgcagtttcacttatgttaaatgttgctcttataccacaaatacattgaaaacttcggatgcatgttgagaaacatgcctttctgtaaaactcaaatataggagctgtgtctacgattcaaagtgaaaacatttggcatgtttgttaattctagctttttggtttaatatcctgtaaggcacgtgagtgtacactttttttttttttaaggatacgggacaattttaagatgtaataccaatactttagaagtttggtcgtgtcgtttgtatgaaaatctgaggctttggtttaaatctttccttgtattgtgatttccatttagatgtattgtactaagtgaaacttgttaaataaatcttccttttaaaaactgga

SEQ ID NO: 2 - DNA, 9756 bp, Homo sapiens
  source 1..9756: mol_type = other DNA
agtagtttgcggcatccggaggagcagcagcagcagtagcggcggcggctggcgcagcgcggagacggacgagcgggcacagacggcagcacagctcccgtgcaccatgcctggcttgcccgggaggcactggcacccgcgtccctagcccggcgctggcccttggcgcgttgcgctccgcagcattccggctgagcgatttcatccgaaatcctaaacctcctaccccaaacccattgcctcctgcttgctcatactccacactcacactccccacaccacacactcactcttcctctctctctcacacattcacacacagcccgagacagacagagaaagcgagacagacctcccgagtgcgcgagcgcccgggaggaagggagcccaacgcagcgcggccagcgcctctccgggaagatgaaatcaagacgctcctgcaagtaaacttcggcggcgacaaaatcgtcgactgtggcagctgctgaggcggaccccagcgacgtgcaactgagctaggagggcttcctcccgcggcggcggcggcggcggcggcggcggcggaccttggagcgacaccccagcccaaacggagagaagagaagcacccggtacccacggcggcggccccgcctctcctggccggtactgttgttgcctccccttccctcttccccattccccgccacccccggccattagaaagaccggggctttgattttcttggacaaattgccatctccccgtttccacaaacttgacgtggagggtcggactgcgttgttaactttgatttcctagacgaaaacgcccgcagaccctcaactgctctgtggggggaactccgctcccacctcggatcctggacagacgcgcccccaagcccttcgaagccagccttagcgcgagggagcgcgctcaagctctggcagcctctctcccctcccccttcctctgctggccccgggaccgccatccgactcaaccctataaccgggtgcccgcggcgcttgccagtaactgactcccgtctgcgacaccccctttctcccgaacaggcctcgaagaggccagtgtggcccccccagaccccggagggcgcattccaagcgggtactctctccgcagtgaactgggagcgctggggggaggtctttgcgcatgccaggtgcacagacagcgagttcgccagcctcagtgccggtgcaccgcggcgggccgggcagtgcctgaaaggctgcgcctcctccgccctctttaccttcgcctcaagtactgtccggactcagcggtgcgtcctccaggccgcgcagacgccgcccgacccgcagccctgggcggctgccgacagtcgcgcccggctaactttgaaggggtaaaactgagtagccgggctggagggagggggctcggggccgcgcggcgctcccttggcccctcggagcccccggggcccccgcgagcccccgccgcgtccgccgcaacctcagcattggagccgccgcgggtgtcccccgcgcgccgccgggccgccctgggcgggacttctcccgcggcctccggggccacggggcgcgcgcaacaggcggcccgagccggcgggaagctggagcgccgacggcgtcggtctcggaggggtgtggagaggcgaggccaggcagagccccgtgcagccatggagtcgctcctgctgccggtgctgctgctgctggccatactgtggacgcaggctgccgccctcattaatctcaagtactcggtagaagaggagcagcgcgccgggacggtgattgccaacgtggccaaagacgcgcgagaggcgggcttcgcgctggacccccggcaggcttcagcctttcgcgtggtgtccaactcggctccacacctagtggacatcaatcccagctctggcctgctggtcaccaagcagaagattgaccgtgatctgctgtgccgccagagccccaagtgcatcatctcgctcgaggtcatgtccagctcaatggaaatctgcgtgataaaggtggagatcaaggacctgaacgacaatgcgcccagtttcccggcagcacagatcgagctggagatctcggaggcagccagccctggcacgcgcatcccgctggacagcgcttacgatccagactcaggaagctttggcgtgcagacttacgagctcacgcccaacgagctgttcggcctggagatcaagacgcgcggcgacggctcccgctttgccgaactcgtggtggaaaagagcctggaccgcgagacgcagtcgcactacagcttccgaatcactgcgctagacggtggcgacccgccgcgcctgggcaccgttggccttagtatcaaggtgaccgactccaatgacaacaacccggtgtttagcgagtccacctacgcggtgagcgtgccagaaaactcgcctcccaacacacccgtcatccgcctcaacgccagcgatccagacgagggcaccaacggccaggtggtctactccttctatggctacgtcaacgaccgcacgcgcgagctctttcagatcgacccgcacagtggcctggtcactgtcactggcgctttagactacgaagaggggcacgtgtacgaactggacgtgcaggctaaggacttggggcccaattccatcccggcacactgcaaggtcaccgtcagcgtgctggacaccaatgacaatccgccggtcatcaacctgctgtcagtcaacagtgagcttgtggaggtcagcgagagcgcccccccgggctacgtgatcgccttggtgcgggtgtctgatcgcgactcaggcctcaatggacgtgtgcagtgccgtttgctgggcaatgtgccctttcgactgcaggaatatgagagcttctccactattctggtggacggacggctggaccgcgagcagcacgaccaatacaacctcacaattcaggcacgcgacggcggcgtgcccatgctgcagagtgccaagtcctttaccgtgctcatcactgacgaaaatgacaaccacccgcacttttccaagccctactaccaggtcattgtgcaggagaacaacacgcctggcgcctatctgctctctgtgtctgctcgcgaccccgacctgggtctcaacggcagtgtctcctaccagatcgtgccgtcgcaggtgcgggacatgcctgtcttcacctatgtctccatcaatcccaactcaggcgacatctacgcgctgcgatcctttaaccacgagcagaccaaggcgttcgaattcaaggtgctggccaaggacggcggccttccctcactgcaaagcaacgctacggtgcgggtcatcatcctcgacgtcaacgacaacaccccggtcatcacagccccacctctgattaacggcactgccgaggtctacataccccgcaactctggcataggctacctggtgactgttgtcaaggcagaagactacgatgagggcgaaaatggccgagtcacctacgacatgaccgagggcgaccgcggcttctttgaaatagaccaggtcaatggcgaagtcagaaccacccgcaccttcggggagagctccaagtcctcctatgagcttatcgtggtggctcacgaccacggcaagacatctctctctgcctctgctctcgtcctaatctacttgtcccctgctctcgatgcccaagagtcaatgggctctgtgaacttgtccttgattttcattattgccctgggctccattgcgggcatcctctttgtaactatgatcttcgtggcaatcaagtgcaagcgagacaacaaagagatccggacctacaactgcagtaattgtttaaccatcacttgtctcctcggctgttttataaaaggacaaaacagcaagtgtctgcattgcatctcggtttctcccattagcgaggagcaagacaaaaagacagaggagaaagtgagcctaaggggaaagagaattgctgagtactcctatgggcatcaaaagaaatcaagcaagaagaaaaaaatcagtaagaatgacatccgcctggtaccccgggatgtggaggagacagacaagatgaacgttgtcagttgctcttccctgacctcctccctcaactattttgactaccaccagcagacgctgcccctgggctgccgccgctctgagagcactttcctgaatgtggagaaccagaatacccgcaacaccagtgctaaccacatctaccatcactctttcaacagccaggggccccagcagcctgacctgattatcaacggtgtgcctctgcctgagactgaaaactattcttttgactccaactacgtgaatagccgagcccatttaatcaagagcagctccaccttcaaggacttagagggcaacagcctgaaggatagtggacatgaggagagtgaccaaactgacagtgagcatgatgtccagcggagcctgtattgtgatactgctgtcaacgatgtgctgaacaccagtgtgacctccatgggatctcagatgcctgatcatgatcagaatgaaggatttcattgccgggaagaatgccggattcttggccactctgacaggtgctggatgccccggaaccccatgcccatccgttccaagtcccctgagcatgtgaggaacatcatcgcgctgtctattgaagctactgctgctgatgtcgaggcttatgacgactgcggccccaccaaacggactttcgcaacctttgggaaagatgtcagcgaccacccggctgaggagaggcctaccctgaaaggcaagaggactgtcgatgtgaccatctgcagccccaaggtcaacagcgttatccgggaggcaggcaatggctgtgaggcgattagccctgtcacctcccccctccacctcaagagctctctgcccaccaagccttccgtgtcttacaccattgccctggctcccccagcccgtgatctggagcagtatgtcaacaatgtcaacaatggccctactcgtccctctgaagctgagccccgtggagctgatagcgagaaagtcatgcatgaggtcagccccattctgaaggaaggtcgcaacaaagagtcccctggtgtgaagcgtctgaaggatatcgttctctaaaccagtctccaggaagaagagaaagaaaccacactggctagtgaagaagcaggagcttcttgttttaattgctcaccaatggttggttcttgagtggctatatttcagagcttttcctaaatgtattgtttataggtgattatcattctgtgacagtcccttgtttcaacaggcagcaggggtgttcagttggagcaaattagctttggcttgagttgttcatggggccttgatgttggggaaacagagacaaattcagttgtgaaaagtattatgtattaagtgtttgaatttatatatttttctatgtcaaaattataatataaattaccattgtttgtggaggattacatttaaaaaagcaaaaagtgaaaaaaaaaagctctggacacctttaataagctcgccactgttttttgtagtgtagacaagttaatggtcatttattgtgtactattcattgattcagtgatgtgaaattgagcccccaaaaggttgtttctgaagctcgagtacttcccaatgccgctactttgctgtggacacccctgctttaaaaacccttttgctagctgtgctattgttgtatttgatattgcaaattgctatgtgtgtggtgatggcaaaaggcttttaaaagttggtgttttctttttcttaaaaaaataataaaactacagacaaaaacaaagtgcaaacaactgagacagcaaatgaatgagcaacaccatgcatatagattgagtacttggagaatactcacgttttttaactcatgtagtgattgctcaccactttgcaaagtatttttcctgcctttcagtggtctgcccaggtccatgatagatcattgcagaaagtcatttttggataggctgctatctaatttgcagttgtcagaaatactgtgctgaaagttttatgacatccatcttttaaattttcaggccctgaacaggaaagttgctcctgaagttatgtttccaggcttagaaattccctctctccaatcatctaaaattgaagatgactgaatctattttaagatctaaattagcatctcttcagacacacacttcctgattcagtgtttcccaatcatgattagaattagactgcaaagcacgtcatgggctgggattgagaactccatgttgctctgtatcttaggcttatatacgtagggatagagtaagcagtacaaagtgtatattttggaaaagacaggtaacaggtaacagcatcgctaatccaattactgtgccttctaaacggagacactcagacacttgaactcatctttttatgactaatagttttttgattaaaaatgtgaacaagaaaatactaaaataaaaatcctttcgatttagccaactctttttgctgtaggctaggaatacaccctttttaaattaacacactgtagatcctttttttctgttttaacattcctctaactcccccatttatcttttgtctattaatattcactagccaacatagtatttttgcagcctaagagttcattatttaaaaataaactaaagaaatatgtcacctttgttctgccttggtcttaagagagttgtttctagagaaacaagttttatggttctgttttcatttgtttcattttttgaaattcaggaatacacagagagaaggcctagaggttagagcactagaatgcaaaagaggatttattttcttaagtttaggtagataagtcagctcttttgaatgttttacttatttttgcctgagttcctagctttacgcattactaggaatattttcttttacaggagggagaagggttttgagggagggggtgggtaaggcagtagggatggggttaggtaggggagaacatttctgaaaaagaacttataatgaagctacaaatccatccttatttcttattcagtgctgaatactacctcatgcaaaatattggtgtggctgcaaatttccctctgaagctgacacaattaaggatgaattaccataacatcttcatttttcctcatttcattgcctctcatacagttttctccctctgatttatttcattttggtagtggattttgaattaagtatttatttctctttgcaagtgactatttgattaacacattaaaaattttttaaaaatttcccctttaagttatgatggctgcctatagaatttagactgtttctcaccctgatccatcctgatattatgttattagctacgatttcaaggtcactttgaagtcagacttcacagttttctacaggtgtatttcttgctatgctcagtgggaaccagggagcagaaaagattgtggaaagtgggaacattaattgtcacgtggctgttgagccagagaggccactggctgccatgcctcttaccagccaagttttaaatggttagagtggcattggcattgattttgcttttctcttgggttttcttcatttcttttcataattcatcttcaagctatagatccatccttcttccctatccaaatactttgttctcagtgggcagtcagatttttttggcttccaaacctcactgagcatgttcagagaattcctctcattaaggaaatatttttccccacaattgtctgatgtatgagtcctctcagaagcacaagtgcatactaggaaggctttgtaaactggttttgtaggcaggctgtacctgcaaataggcattctgagcaaggatcctaaatttccctttggctcatgtcccccccgccccaccccaccttctcctttttggcatgtctggtgaaaagctacaaggttcacagggccaaatattctcccttttgttctctaaacatgttgcacacataaactcaagggagtgatttcggttcctaagtcctagggagttctattccatttattctgatgtcccttttaaggaggagttggtgttgaagtgtgacatgctttaatgataactgctttggcaattccccagacaggtatacttcttcatccaaaattgcacctaaggtgttcatttagacaaaattgttcgcttagtgatccactgatctctaatcagaaatatttttaaaaaatacaatttagatctctgaaaattggaaccactcaagagtatcatcttgatagtgttacttcagcatatgactgactgattttatgaagggagtttagctgacttttccctgtagccctatcatattaaaaacaaaaagtggtccacttgaagcagcaaggacaaagggcagtttagggagaggcagggtgaaggggatagctcgattgggcatccagagtggaaaaggattattattgtacataatagcagttctggctggaattccaatccactttctggggatcattttgatgttgaatgttcccccaaagcattctgacaagtttggtccacttggctctttaccccgggtaataagacccttattgactttgaaaaactggcataaatgagcttaagaaatccattgtatacagtcctagtgtttgccttcttggtaaaattacatttaaggatgtcatttcatgagtcaaattgtatctgctgacactgaagtgtattgagatgttgttctttagtttaagtatgtaacaaagtaatattcaaaggggatgtttgggggtggggctgggaccgggtggctttatattgataatgaaatattccagagcaaaacacattgtcctaatgaagttgttgcagctctcttcgttttcttgtgccgctggaattggcttgtgggtcacttgttgtgcaaaagtgaattaatttcaaataggaatcatattgcttttgtgaaacaaagaaaatttaccaccttgtctgatacatagattggaaggcgctcttggtaaagaataacccacaaacacctgatcttgattggaaagcattgtaggtcattagggaaaactgatgatgctgactgttgctccagctgtgttggcaattcaagatgaccttgttactaactgaaagttgctgtcaaccttagcaaacaaaccagctaacaccattacagcactggttaagtagatttttactggcactggctggcgttcagctgaggaaggctaggatacagactttaatgaccaagagccagactagctcgagctagaaatcagcaacaaatacctccttgtgctatttgaatgctaaggtattagttgcaataggcttacgccttcgctcctctagtgaggggaatgagcctgatgacactattactgtttacctgtcaaaccagggtggaaatttagagggatgcaagtacagtatgtctcattaagcagtctctgttgctgattatatgaagataaaattgttgtatatgctgatctgtatgttatgtacattttcacagtgattgaatcattgtaaaaagaaaaaagacaaaaaagaatcaccactgtctattttatgaagatgataaagcagctttattaaattattacagttatgtttcaaatggtgttatctgccacattgtttccttttatctggtggttctctgtttccccctttaaatgtgttttctctgcttcaaaaatttaatgcattttagtattaaaggctattatggaaaaagta

SEQ ID NO: 3 - DNA, 20 bp, synthetic construct
  source 1..20: mol_type = other DNA
tagacgacgaccagaactac

SEQ ID NO: 4 - DNA, 20 bp, synthetic construct
  misc_feature 1..20: note = Synthetic
  source 1..20: mol_type = other DNA
ataccgggtaattttcctcc

SEQ ID NO: 5 - DNA, 20 bp, synthetic construct
  misc_feature 1..20: note = Synthetic
  source 1..20: mol_type = other DNA
atcagagaagaagcgcatca

SEQ ID NO: 6 - DNA, 20 bp, synthetic construct
  misc_feature 1..20: note = Synthetic
  source 1..20: mol_type = other DNA
cttcctcttcagattcaagt

SEQ ID NO: 7 - DNA, 20 bp, synthetic construct
  misc_feature 1..20: note = Synthetic
  source 1..20: mol_type = other DNA
gtatgcttcaacacccaatc

SEQ ID NO: 8 - DNA, 20 bp, synthetic construct
  misc_feature 1..20: note = Synthetic
  source 1..20: mol_type = other DNA
aactgaacaatctcttcctt

SEQ ID NO: 9 - DNA, 20 bp, synthetic construct
  misc_feature 1..20: note = Synthetic
  source 1..20: mol_type = other DNA
tttaaggccttctcagctat

SEQ ID NO: 10 - DNA, 20 bp, synthetic construct
  misc_feature 1..20: note = Synthetic
  source 1..20: mol_type = other DNA
tttcgagggggatcatagtg

SEQ ID NO: 11 - DNA, 20 bp, synthetic construct
  misc_feature 1..20: note = Synthetic
  source 1..20: mol_type = other DNA
taccctccaccataggcacc

SEQ ID NO: 12 - DNA, 20 bp, synthetic construct
  misc_feature 1..20: note = Synthetic
  source 1..20: mol_type = other DNA
agtaattgaggtctcttcca

SEQ ID NO: 13 - DNA, 20 bp, synthetic construct
  misc_feature 1..20: note = Synthetic
  source 1..20: mol_type = other DNA
attctcagtggctctgtaag

SEQ ID NO: 14 - DNA, 20 bp, synthetic construct
  misc_feature 1..20: note = Synthetic
  source 1..20: mol_type = other DNA
tgccacagcatcttcatgag

SEQ ID NO: 15 - DNA, 20 bp, synthetic construct
  misc_feature 1..20: note = Synthetic
  source 1..20: mol_type = other DNA
aaaagttctacatagctgtg

SEQ ID NO: 16 - DNA, 20 bp, synthetic construct
  misc_feature 1..20: note = Synthetic
  source 1..20: mol_type = other DNA
ccaccacttgcccctgctgt

SEQ ID NO: 17 - DNA, 20 bp, synthetic construct
  misc_feature 1..20: note = Synthetic
  source 1..20: mol_type = other DNA
taaccaccactcagctgctg

SEQ ID NO: 18 - DNA, 20 bp, synthetic construct
  misc_feature 1..20: note = Synthetic
  source 1..20: mol_type = other DNA
taagcaaggtttgactgata

SEQ ID NO: 19 - DNA, 20 bp, synthetic construct
  misc_feature 1..20: note = Synthetic
  source 1..20: mol_type = other DNA
ttcttaaaaaaggagaaaga

SEQ ID NO: 20 - DNA, 20 bp, synthetic construct
  misc_feature 1..20: note = Synthetic
  source 1..20: mol_type = other DNA
aactaacttgaggaaagcta

SEQ ID NO: 21 - DNA, 20 bp, synthetic construct
  misc_feature 1..20: note = Synthetic
  source 1..20: mol_type = other DNA
ttcactttgaatcgtagaca

SEQ ID NO: 22 - DNA, 20 bp, synthetic construct
  misc_feature 1..20: note = Synthetic
  source 1..20: mol_type = other DNA
aaaggaagatttatttaaca

SEQ ID NO: 23 - DNA, 20 bp, synthetic construct
  misc_feature 1..20: note = Synthetic
  source 1..20: mol_type = other DNA
ggtttaggatttcggatgaa

SEQ ID NO: 24 - DNA, 20 bp, synthetic construct
  misc_feature 1..20: note = Synthetic
  source 1..20: mol_type = other DNA
gggagtcagttactggcaag

SEQ ID NO: 25 - DNA, 20 bp, synthetic construct
  misc_feature 1..20: note = Synthetic
  source 1..20: mol_type = other DNA
caccggcagcaggagcgact

SEQ ID NO: 26 - DNA, 20 bp, synthetic construct
  misc_feature 1..20: note = Synthetic
  source 1..20: mol_type = other DNA
gtcaatcttctgcttggtga

SEQ ID NO: 27 - DNA, 20 bp, synthetic construct
  misc_feature 1..20: note = Synthetic
  source 1..20: mol_type = other DNA
cgttgggcgtgagctcgtaa

SEQ ID NO: 28 - DNA, 20 bp, synthetic construct
  misc_feature 1..20: note = Synthetic
  source 1..20: mol_type = other DNA
gtgttgggaggcgagttttc

SEQ ID NO: 29 - DNA, 20 bp, synthetic construct
  misc_feature 1..20: note = Synthetic
  source 1..20: mol_type = other DNA
gtccagcacgctgacggtga

SEQ ID NO: 30 - DNA, 20 bp, synthetic construct
  misc_feature 1..20: note = Synthetic
  source 1..20: mol_type = other DNA
tgaggttgtattggtcgtgc

SEQ ID NO: 31 - DNA, 20 bp, synthetic construct
  misc_feature 1..20: note = Synthetic
  source 1..20: mol_type = other DNA
gatggagacataggtgaaga

SEQ ID NO: 32 - DNA, 20 bp, synthetic construct
  misc_feature 1..20: note = Synthetic
  source 1..20: mol_type = other DNA
agtcaccaggtagcctatgc

SEQ ID NO: 33 - DNA, 20 bp, synthetic construct
  misc_feature 1..20: note = Synthetic
  source 1..20: mol_type = other DNA
ttgactcttgggcatcgaga

SEQ ID NO: 34 - DNA, 20 bp, synthetic construct
  misc_feature 1..20: note = Synthetic
  source 1..20: mol_type = other DNA
tcctctgtctttttgtcttg

SEQ ID NO: 35 - DNA, 20 bp, synthetic construct
  misc_feature 1..20: note = Synthetic
  source 1..20: mol_type = other DNA
ctggttctccacattcagga

SEQ ID NO: 36 - DNA, 20 bp, synthetic construct
  misc_feature 1..20: note = Synthetic
  source 1..20: mol_type = other DNA
ctcactgtcagtttggtcac

SEQ ID NO: 37 - DNA, 20 bp, synthetic construct
  misc_feature 1..20: note = Synthetic
  source 1..20: mol_type = other DNA
catcagcagcagtagcttca

SEQ ID NO: 38 - DNA, 20 bp, synthetic construct
  misc_feature 1..20: note = Synthetic
  source 1..20: mol_type = other DNA
agccagggcaatggtgtaag

SEQ ID NO: 39 - DNA, 20 bp, synthetic construct
  misc_feature 1..20: note = Synthetic
  source 1..20: mol_type = other DNA
ggaaaagctctgaaatatag

SEQ ID NO: 40 - DNA, 20 bp, synthetic construct
  misc_feature 1..20: note = Synthetic
  source 1..20: mol_type = other DNA
cagcccatgacgtgctttgc

SEQ ID NO: 41 - DNA, 20 bp, synthetic construct
  misc_feature 1..20: note = Synthetic
  source 1..20: mol_type = other DNA
ataggcagccatcataactt

SEQ ID NO: 42 - DNA, 20 bp, synthetic construct
  misc_feature 1..20: note = Synthetic
  source 1..20: mol_type = other DNA
tatccccttcaccctgcctc

SEQ ID NO: 43 - DNA, 20 bp, synthetic construct
  misc_feature 1..20: note = Synthetic
  source 1..20: mol_type = other DNA
  misc_feature 1..5: note = RNA
  misc_feature 6..15: note = DNA
  misc_feature 16..20: note = RNA
  modified_base 1..20: mod_base = OTHER; note = phosphorothioate bond
  modified_base 1..5: mod_base = OTHER; note = 2'-O-methoxyethyl
  modified_base 16..20: mod_base = OTHER; note = 2'-O-methoxyethyl
tagacgacgaccagaactac

SEQ ID NO: 44 - DNA, 20 bp, synthetic construct
  source 1..20: mol_type = other DNA
  misc_feature 6..15: note = DNA
  misc_feature 1..5: note = RNA
  misc_feature 16..20: note = RNA
  modified_base 1..20: mod_base = OTHER; note = phosphorothioate
  modified_base 1..5: mod_base = OTHER; note = 2'-O-methoxyethyl
  modified_base 16..20: mod_base = OTHER; note = 2'-O-methoxyethyl
ataccgggtaattttcctcc

SEQ ID NO: 45 - DNA, 20 bp, synthetic construct
  source 1..20: mol_type = other DNA
  modified_base 1..20: mod_base = OTHER; note = phosphorothioate
  modified_base 1..5: mod_base = OTHER; note = 2'-O-methoxyethyl
  misc_feature 6..15: note = DNA
  misc_feature 1..5: note = RNA
  modified_base 16..20: mod_base = OTHER; note = 2'-O-methoxyethyl
  misc_feature 16..20: note = RNA
atcagagaagaagcgcatca

SEQ ID NO: 46 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
  misc_feature 6..15: note = DNA
  misc_feature 1..5: note = RNA
  misc_feature 16..20: note = RNA
  modified_base 1..20: mod_base = OTHER; note = phosphorothioate
cttcctcttcagattcaagt

SEQ ID NO: 47 - DNA, 20 bp, synthetic construct
  source 1..20: mol_type = other DNA
  modified_base 1..20: mod_base = OTHER; note = phosphorothioate
  modified_base 16..20: mod_base = OTHER; note = 2'-O-methoxyethyl
  misc_feature 6..15: note = DNA
  misc_feature 1..5: note = RNA
  misc_feature 16..20: note = RNA
  modified_base 1..5: mod_base = OTHER; note = 2'-O-methoxyethyl
gtatgcttcaacacccaatc

SEQ ID NO: 48 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
  misc_feature 1..5: note = RNA
  misc_feature 6..15: note = DNA
  misc_feature 16..20: note = RNA
  modified_base 1..20: mod_base = OTHER; note = phosphorothioate
aactgaacaatctcttcctt

SEQ ID NO: 49 - DNA, 20 bp, synthetic construct
  source 1..20: mol_type = other DNA
  modified_base 1..20: mod_base = OTHER; note = phosphorothioate
  modified_base 1..5: mod_base = OTHER; note = 2'-O-methoxyethyl
  misc_feature 6..15: note = DNA
  misc_feature 1..5: note = RNA
  misc_feature 16..20: note = RNA
  modified_base 16..20: mod_base = OTHER; note = 2'-O-methoxyethyl
tttaaggccttctcagctat

SEQ ID NO: 50 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
  misc_feature 6..15: note = DNA
  misc_feature 1..5: note = RNA
  misc_feature 16..20: note = RNA
  modified_base 1..20: mod_base = OTHER; note = phosphorothioate
tttcgagggggatcatagtg

SEQ ID NO: 51 - DNA, 20 bp, synthetic construct
  source 1..20: mol_type = other DNA
  modified_base 1..20: mod_base = OTHER; note = phosphorothioate
  modified_base 1..5: mod_base = OTHER; note = 2'-O-methoxyethyl
  misc_feature 6..15: note = DNA
  misc_feature 16..20: note = RNA
  misc_feature 1..5: note = RNA
  modified_base 16..20: mod_base = OTHER; note = 2'-O-methoxyethyl
taccctccaccataggcacc

SEQ ID NO: 52 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
  misc_feature 1..5: note = RNA
  misc_feature 6..15: note = DNA
  misc_feature 16..20: note = RNA
  modified_base 1..20: mod_base = OTHER; note = phosphorothioate
agtaattgaggtctcttcca

SEQ ID NO: 53 - DNA, 20 bp, synthetic construct
  source 1..20: mol_type = other DNA
  modified_base 1..20: mod_base = OTHER; note = phosphorothioate
  modified_base 1..5: mod_base = OTHER; note = 2'-O-methoxyethyl
  misc_feature 6..15: note = DNA
  misc_feature 1..5: note = RNA
  misc_difference 16..20: note = RNA
  modified_base 16..20: mod_base = OTHER; note = 2'-O-methoxyethyl
attctcagtggctctgtaag

SEQ ID NO: 54 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
  misc_feature 6..15: note = DNA
  misc_feature 1..5: note = RNA
  misc_feature 16..20: note = RNA
  modified_base 1..20: mod_base = OTHER; note = phosphorothioate
tgccacagcatcttcatgag

SEQ ID NO: 55 - DNA, 20 bp, synthetic construct
  source 1..20: mol_type = other DNA
  modified_base 1..20: mod_base = OTHER; note = phosphorothioate
  modified_base 1..5: mod_base = OTHER; note = 2'-O-methoxyethyl
  misc_feature 6..15: note = DNA
  misc_feature 1..5: note = RNA
  misc_feature 16..20: note = RNA
  modified_base 16..20: mod_base = OTHER; note = 2'-O-methoxyethyl
tgccacagcatcttcatgag

SEQ ID NO: 56 - DNA, 20 bp, synthetic construct
  source 1..20: mol_type = other DNA
  modified_base 1..5: mod_base = OTHER; note = 2'-O-methoxyethyl
  modified_base 1..20: mod_base = OTHER; note = phosphorothioate
  misc_feature 1..5: note = RNA
  misc_feature 6..15: note = DNA
  modified_base 16..20: mod_base = OTHER; note = 2'-O-methoxyethyl
ccaccacttgcccctgctgt

SEQ ID NO: 57 - DNA, 20 bp, synthetic construct
  source 1..20: mol_type = other DNA
  modified_base 1..20: mod_base = OTHER; note = phosphorothioate
  modified_base 1..5: mod_base = OTHER; note = 2'-O-methoxyethyl
  misc_feature 1..5: note = RNA
  misc_feature 6..15: note = DNA
  misc_feature 16..20: note = RNA
  modified_base 16..20: mod_base = OTHER; note = 2'-O-methoxyethyl
taaccaccactcagctgctg

SEQ ID NO: 58 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
  misc_feature 1..5: note = RNA
  misc_feature 6..15: note = DNA
  misc_feature 16..20: note = RNA
  modified_base 1..20: mod_base = OTHER; note = phosphorothioate
taagcaaggtttgactgata

SEQ ID NO: 59 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
  misc_feature 6..15: note = DNA
  misc_feature 1..5: note = RNA
  misc_feature 16..20: note = RNA
  modified_base 1..20: mod_base = OTHER; note = phosphorothioate
ttcttaaaaaaggagaaaga

SEQ ID NO: 60 - DNA, 20 bp, synthetic construct
  source 1..20: mol_type = other DNA
  modified_base 1..20: mod_base = OTHER; note = phosphorothioate
  modified_base 1..5: mod_base = OTHER; note = 2'-O-methoxyethyl
  misc_feature 6..15: note = DNA
  misc_feature 1..5: note = RNA
  misc_feature 16..20: note = RNA
  modified_base 16..20: mod_base = OTHER; note = 2'-O-methoxyethyl
aactaacttgaggaaagcta

SEQ ID NO: 61 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
ttcactttgaatcgtagaca

SEQ ID NO: 62 - DNA, 20 bp, synthetic construct
  source 1..20: mol_type = other DNA
  misc_feature 1..5: note = RNA
  misc_feature 6..15: note = DNA
  misc_feature 16..20: note = RNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
aaaggaagatttatttaaca

SEQ ID NO: 63 - DNA, 20 bp, synthetic construct
  misc_feature 1..5: note = RNA
  source 1..20: mol_type = other DNA
  misc_feature 6..15: note = DNA
  misc_feature 16..20: note = RNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
ggtttaggatttcggatgaa

SEQ ID NO: 64 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
gggagtcagttactggcaag

SEQ ID NO: 65 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
caccggcagcaggagcgact

SEQ ID NO: 66 - DNA, 20 bp, synthetic construct
  misc_feature 1..5: note = RNA
  misc_feature 6..15: note = DNA
  misc_feature 16..20: note = DNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
gtcaatcttctgcttggtga

SEQ ID NO: 67 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
cgttgggcgtgagctcgtaa

SEQ ID NO: 68 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
gtgttgggaggcgagttttc

SEQ ID NO: 69 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
gtccagcacgctgacggtga

SEQ ID NO: 70 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
tgaggttgtattggtcgtgc

SEQ ID NO: 71 - DNA, 20 bp, synthetic construct
  misc_feature 1..5: note = RNA
  misc_feature 6..15: note = DNA
  misc_feature 6..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
gatggagacataggtgaaga

SEQ ID NO: 72 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
agtcaccaggtagcctatgc

SEQ ID NO: 73 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
ttgactcttgggcatcgaga

SEQ ID NO: 74 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
tcctctgtctttttgtcttg

SEQ ID NO: 75 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
ctggttctccacattcagga

SEQ ID NO: 76 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
ctcactgtcagtttggtcac

SEQ ID NO: 77 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
catcagcagcagtagcttca

SEQ ID NO: 78 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
agccagggcaatggtgtaag

SEQ ID NO: 79 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
ggaaaagctctgaaatatag

SEQ ID NO: 80 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
cagcccatgacgtgctttgc

SEQ ID NO: 81 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
ataggcagccatcataactt

SEQ ID NO: 82 - DNA, 20 bp, synthetic construct
  misc_feature 1..5: note = RNA
  misc_feature 6..15: note = DNA
  misc_feature 16..20: note = RNA
  source 1..20: mol_type = other DNA
  modified_base 1..5: mod_base = OTHER; note = 2'-O-methoxyethyl
  modified_base 1..20: mod_base = OTHER; note = phosphorothioate
  modified_base 16..20: mod_base = OTHER; note = 2'-O-methoxyethyl
tatccccttcaccctgcctc

SEQ ID NO: 83 - DNA, 2199 bp, Homo sapiens
  source 1..2199: mol_type = other DNA
catttcgtcttagccacgcagaagtcgcgtgtctagtttgtttcgacgccggaccgcgtaagagacgatgatgttgggcacggaaggtggagagggattcgtggtgaaggtccggggcttgccctggtcttgctcggccgatgaagtgcagaggtttttttctgactgcaaaattcaaaatggggctcaaggtattcgtttcatctacaccagagaaggcagaccaagtggcgaggcttttgttgaacttgaatcagaagatgaagtcaaattggccctgaaaaaagacagagaaactatgggacacagatatgttgaagtattcaagtcaaacaacgttgaaatggattgggtgttgaagcatactggtccaaatagtcctgacacggccaatgatggctttgtacggcttagaggacttccctttggatgtagcaaggaagaaattgttcagttcttctcagggttggaaatcgtgccaaatgggataacattgccggtggacttccaggggaggagtacgggggaggccttcgtgcagtttgcttcacaggaaatagctgaaaaggctctaaagaaacacaaggaaagaatagggcacaggtatattgaaatctttaagagcagtagagctgaagttagaactcattatgatccaccacgaaagcttatggccatgcagcggccaggtccttatgacagacctggggctggtagagggtataacagcattggcagaggagctggctttgagaggatgaggcgtggtgcttatggtggaggctatggaggctatgatgattacaatggctataatgatggctatggatttgggtcagatagatttggaagagacctcaattactgtttttcaggaatgtctgatcacagatacggggatggtggctctactttccagagcacaacaggacactgtgtacacatgcggggattaccttacagagctactgagaatgacatttataattttttttcaccgctcaaccctgtgagagtacacattgaaattggtcctgatggcagagtaactggtgaagcagatgtcgagttcgcaactcatgaagatgctgtggcagctatgtcaaaagacaaagcaaatatgcaacacagatatgtagaactcttcttgaattctacagcaggagcaagcggtggtgcttacgaacacagatatgtagaactcttcttgaattctacagcaggagcaagcggtggtgcttatggtagccaaatgatgggaggcatgggcttgtcaaaccagtccagctacgggggcccagccagccagcagctgagtgggggttacggaggcggctacggtggccagagcagcatgagtggatacgaccaagttttacaggaaaactccagtgattttcaatcaaacattgcataggtaaccaaggagcagtgaacagcagctactacagtagtggaagccgtgcatctatgggcgtgaacggaatgggagggttgtctagcatgtccagtatgagtggtggatggggaatgtaattgatcgatcctgatcactgactcttggtcaaccttttttttttttttttttttttctttaagaaaacttcagtttaacagtttctgcaatacaagcttgtgatttatgcttactctaagtggaaatcaggattgttatgaagacttaaggcccagtatttttgaatacaatactcatctaggatgtaacagtgaagctgagtaaactataactgttaaacttaagttccagcttttctcaagttagttataggatgtacttaagcagtaagcgtatttaggtaaaagcagttgaattatgttaaatgttgccctttgccacgttaaattgaacactgttttggatgcatgttgaaagacatgcttttatttttttgtaaaacaatataggagctgtgtctactattaaaagtgaaacattttggcatgtttgttaattctagtttcatttaataacctgtaaggcacgtaagtttaagctttttttttttttaagttaatgggaaaaatttgagacgcaataccaatacttaggattttggtcttggtgtttgtatgaaattctgaggccttgatttaaatctttcattgtattgtgatttccttttaggtatattgcgctaagtgaaacttgtcaaataaatcctccttttaaaaactgca

SEQ ID NO: 84 - DNA, 20 bp, synthetic construct
  source 1..20: mol_type = other DNA
cacttctcgtttgcctcctc

SEQ ID NO: 85 - DNA, 20 bp, synthetic construct
  source 1..20: mol_type = other DNA
caccatcctcgtctgtgatg

SEQ ID NO: 86 - DNA, 22 bp, synthetic construct
  source 1..22: mol_type = other DNA
tgcctattcaactgaaggctcc

SEQ ID NO: 87 - DNA, 22 bp, synthetic construct
  source 1..22: mol_type = other DNA
ggcagaaggaatacaaggcaac

SEQ ID NO: 88 - DNA, 24 bp, synthetic construct
  source 1..24: mol_type = other DNA
caacagcttgagactggcaagtga

SEQ ID NO: 89 - DNA, 25 bp, synthetic construct
  source 1..25: mol_type = other DNA
ctagaattgctggatcgaccctggg

SEQ ID NO: 90 - DNA, 20 bp, synthetic construct
  source 1..20: mol_type = other DNA
gagaagggggaagagaagga

SEQ ID NO: 91 - DNA, 20 bp, synthetic construct
  source 1..20: mol_type = other DNA
tcagttatgcgccacttcag

SEQ ID NO: 92 - DNA, 20 bp, synthetic construct
  source 1..20: mol_type = other DNA
tttggagagcacccctttta

SEQ ID NO: 93 - DNA, 21 bp, synthetic construct
  source 1..21: mol_type = other DNA
caatccacgggtcataggtaa

SEQ ID NO: 94 - DNA, 17 bp, synthetic construct
  source 1..17: mol_type = other DNA
cgtggacatccgcaaag

SEQ ID NO: 95 - DNA, 17 bp, synthetic construct
  source 1..17: mol_type = other DNA
ggaaggtggacagcgag